(19) United States
(12) Patent Application Publication
(10) Pub. No.: US 2025/0354096 A1
(43) Pub. Date: Nov. 20, 2025

(54) IN VITRO DEVICE FOR VASCULARIZED MICROFLUIDIC MODELING OF OSTEOCHONDRAL UNIT

(71) Applicant: CFD Research Corporation, Huntsville, AL (US)

(72) Inventors: Kevin Daniel Roehm, Huntsville, AL (US); Ashley Gilbert Helser, Huntsville, AL (US); Katie Lauren Dietz, Huntsville, AL (US); Balabhaskar Prabhakarpandian, Madison, AL (US)

(21) Appl. No.: 18/665,244

(22) Filed: May 15, 2024

(51) Int. Cl. C12M 3/00; C12M 1/00; C12M 3/06; C12N 5/077; G06T 7/00

(52) U.S. Cl. CPC C12M 21/08; C12M 23/16; C12M 29/04; C12N 5/0655; G06T 7/0012; C12N 2506/1346; G06T 2207/30024

(57) ABSTRACT

A method of differentiating cells can include: providing the in vitro OC device of one of the embodiments; introducing first human mesenchymal stem cell into the cartilage chamber; introducing a chondrogenic differentiation medium into the cartilage chamber with the first human mesenchymal stem cells; incubating the first human mesenchymal stem cells with the chondrogenic differentiation medium sufficiently to differentiate into at least one of chondrocyte cells, chondroblast cells, and/or chondroclast cells; incubating second human mesenchymal stem cells with osteogenic differentiation medium sufficiently to differentiate into at least one of osteoblast cells, osteoclast cells, and/or osteocyte cells; introducing the differentiated at least one of osteoblast cells, osteoclast cells, and/or osteocyte cells into the bone chamber; and introducing vascular endothelial cells into the vasculature chamber.

### U.S. GOVERNMENT RIGHTS

This invention was made with government support under 1R43AR072169-01 awarded by the National Institutes of Health. The government has certain rights in the invention.

### BACKGROUND

### Field

This invention relates to the fields of physiology and microfluidics applied to the osteochondral unit of the articular cartilage and subchondral bone, such as in a joint. More specifically, the present invention pertains to microfluidic systems that mimic the structure, dimensions, fluid flow, and physiological function of the osteochondral unit of the cartilage and bone, as well as differentiation of human mesenchymal stem cells (hMSCs) into bone cells and cartilage cells, and in vitro assays related to osteoarthritis and treatments thereof.

### Description of Related Art

It is known that osteoarthritis (OA) is a common degenerative joint disease that is a leading cause of chronic pain [1]. The World Health Organization estimates that 9.6% of men and 18.0% of women over 60 years of age suffer from symptomatic OA worldwide [2,3]. OA affects over 32.5 million adults in the United States alone [4]. OA is mainly characterized by the progressive erosion, calcification, and vascular invasion of articular cartilage, thickening of the subchondral bone, and inflammation of the synovium [5-8]. Despite its prevalence, treatments for OA are limited to pain management and surgical intervention (e.g., joint replacement). Currently, there are no effective pharmacological therapies to slow progression of the disease, and no therapies to reverse its course [9]. The lack of treatment is mainly due to a limited understanding of the underlying mechanisms and etiology of OA [10]. Traditionally, OA was thought to be limited to articular cartilage, as a simple consequence of wear and tear affecting this tissue [11].

However, recent findings revealed that OA is a disease of the whole joint, characterized by significant changes in both articular cartilage and subchondral bone, and the interactions between these tissues plays a significant role in the disease [12-15]. For instance, vascular invasion of the articular cartilage from the subchondral bone takes place during the disease, thus enhancing the crosstalk between bone and cartilage, and likely leading to an increased transport of cytokines.

In addition, the direct causes of OA are still unclear, although there are multiple known risk factors such as age, congenital predisposing factors, and other metabolic disorders, such as diabetes [16,17]. Recent evidence showed that elevation of lipopolysaccharide (LPS; an outer-membrane component of gram-negative bacteria) and other pro-inflammatory molecules, such as interleukins and tumor necrosis factors alpha, contribute to the inflammatory response of OA [1,18-21].

Overall, the need to understand the etiology of OA, as well as the increasing evidence of the interplay between subchondral bone and articular cartilage, highlights the urgent demand for realistic models of the osteochondral (OC) unit that mimic articular cartilage and vascularized subchondral bone as a platform to investigate OA and potential therapeutic candidates. In this context, several in vitro, ex vivo (i.e., explants), and in vivo animal models including horse, dog, mouse [22], rat [23], rabbit, and Guinea Pig [24], have been developed. Animal models have the advantage of mimicking the entire joint and can be used to explore aging, obesity, and genetics [9,24]. However, animal models have limited predictivity on the effects of drugs in humans [25-27] and cannot distinguish individual contributions to complex tissues interactions. Moreover, animal models of naturally occurring OA require extended experimental time, thus increasing the costs of studies.

Explants (e.g., OC unit isolated from osteoarthritic patients during joint replacement surgeries) are realistic 3D models from actual patients; however, they lack consistency between donors and suffer from a limited supply, thus limiting the possibilities of screening studies. In addition, because they represent advanced OA, human explants are poor models for studying the early stage of OA development. On the other hand, 2D in vitro models are repeatable, but are only able to study signaling mechanisms of a few cell types and lack in vivo morphology within a 3D structure, which often alters cellular activity and phenotypes [28].

There are 3D in vitro models based on engineered tissues that comprise both bone and cartilage phases, which provide biomimicry, repeatability, and greater complexity with lower cost. Notwithstanding the need for 3D OA models, however, few such studies are found in literature. For example, Stüdle et al. developed a 3D model to study ectopic OC tissue in which bone marrow stem cells (BMSCs) seeded in a poly (ethylene glycol) (PEG) scaffold are layered with PEG-seeded nasal chondrocytes. This 3D construct results in a calcified bottom layer of BMSCs and a cartilaginous top layer of chondrocytes producing a static 3D coculture model.

Previously, a microphysiological system bioreactor was created to be able to maintain two separate compartments (e.g., chondral and osseous), to develop a 3D OC model of OA where bone and cartilage are in contact and can signal to each other, while each being directly exposed only to tissue-specific culture media [30]. The described bioreactor generates a continuous 3D environment with distinct chondral and osseous zones by controlling the media in each compartment. This system was previously used to introduce an OA-like catabolic response in a OC construct by exposing the osseous and/or chondral phases to interleukin-beta 1 as proinflammatory molecules [1]. Although cutting-edge, these studies do not meet the need for an in vitro platform with real-time monitoring and analysis capability of vascularized bone-cartilage interactions, which is necessary to mechanistically investigate the etiology and progression of OA.

Therefore, it would be advantageous to develop an innovative 3D microfluidic device able to provide a real-time visualization and quantification of vasculature, bone, and cartilage of an OC unit, that are individually co-cultured in separate chambers with porous interconnections to allow for tissue-tissue crosstalk.

### SUMMARY

In some embodiments, a microfluidic in vitro osteochondral (OC) device can include: a synovial chamber; a cartilage chamber adjacent to and porously coupled with the synovial chamber; a bone chamber adjacent to and porously coupled with the cartilage chamber; a vasculature circulation chamber adjacent to and porously coupled with the bone chamber; wherein a first porous wall is positioned between the synovial chamber and the cartilage chamber, a second porous wall is positioned between the cartilage chamber and the bone chamber, and a third porous wall is positioned between the bone chamber and the vasculature circulation chamber. The device is configured as a microfluidic in vitro model of an in vivo osteochondral region of a subject. That is, the device mimics a real live osteochondral region, such as in a live joint of a living organism, such as a mammal (e.g., human).

In some embodiments, a microfluidic in vitro OC system can include the microfluidic in vitro OC device of one of the embodiments, and at least one pump configured for pumping fluid through the microfluidic in vitro OC device. The pump can be a single pass pump or a recirculating pump, or any other type of pump.

In some embodiments, a microfluidic in vitro OC system can include: the microfluidic in vitro OC device of one of the embodiments, at least one camera device configured to be positioned to image at least one of the synovial chamber, cartilage chamber, bone chamber, or vasculature circulation chamber, and a computing system operably coupled with the at least one camera device to receive image data.

In some embodiments, a method of studying an osteochondral environment can include: providing the microfluidic in vitro OC device of one of the embodiments having the cells therein; measuring a first condition of the microfluidic in vitro OC device at a first time point; measuring a second condition of the in vitro OC device at a subsequent time point; and determining a change in condition of the in vitro OC device from the first condition to the second condition.

In some embodiments, a method of studying transport of a test agent across an OC region can include: providing the microfluidic in vitro OC device of one of the embodiments having the cell cultures; providing a test agent to an input chamber selected from the synovial chamber, cartilage chamber, bone chamber, or vasculature circulation chamber; and monitoring transport of the test agent across at least one of the cartilage chamber or the bone chamber.

In some embodiments, a method of differentiating cells can include: providing the in vitro OC device of one of the embodiments; introducing first human mesenchymal stem cell into the cartilage chamber; introducing a chondrogenic differentiation medium into the cartilage chamber with the first human mesenchymal stem cells; incubating the first human mesenchymal stem cells with the chondrogenic differentiation medium sufficiently to differentiate into at least one of chondrocyte cells, chondroblast cells, and/or chondroclast cells; incubating second human mesenchymal stem cells with osteogenic differentiation medium sufficiently to differentiate into at least one of osteoblast cells, osteoclast cells, and/or osteocyte cells; introducing the differentiated at least one of osteoblast cells, osteoclast cells, and/or osteocyte cells into the bone chamber; and introducing vascular endothelial cells into the vasculature chamber.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A includes a top view of the in vitro osteochondral device having a microfluidic network that includes a vasculature chamber, bone chamber, cartilage chamber, and fluidic chamber.

FIG. 1B is an enlarged portion of the assay region of FIG. 1A, which shows more details of the vasculature chamber, bone chamber, cartilage chamber, and fluidic chamber.

FIG. 1C shows an enlargement of a porous wall separating the cartilage chamber and the fluidic chamber.

FIG. 1D shows a side view of a schematic representation of the in vitro OC device of FIGS. 1A-1C.

FIG. 1E shows cameras that can be placed outside of the glass slide, outside the substrate, or embedded in the substrate (e.g., in etched regions).

FIG. 1F shows an exemplary system that includes the in vitro OC device.

FIG. 2A includes an image that shows the validation of pore size and barrier integrity with fluorescent polystyrene beads (blue and red in outer channels are 2 μm, green are 4 μm in inner channel).

FIG. 2B includes an image that is a zoom in of the red 2 μm particles passing through the barrier of FIG. 2A.

FIG. 2C is a zoom in of blue 2 μm particles passing through the barrier of FIG. 2A.

FIG. 3 includes a micrograph showing the OC device having channels comprised of synovial fluid, differentiated chondrocyte, chondroblasts, and/or chondroblasts cell culture, differentiated osteocyte, osteoclasts, and/or osteoblasts cell culture, and vasculature endothelial cell culture.

FIG. 4A includes images that show staining for osteocalcin and hydroxyapatite in growth medium and osteogenic medium, which shows the differentiation into bone-like environment.

FIG. 4B includes a graph that shows that osteogenic differentiation medium increases the concentration of hydroxyapatite, which shows the differentiation into bone-like environment.

FIG. 5 includes images that show the differentiation increased from day 3 to day 20 with the chondrogenic differentiation medium, as shown by increased Aggrecan and Collagen II.

FIGS. 6A-6B includes images of cultures in the OC device that demonstrate the morphology of osteoblast and HUVEC that are side-by-side, which permits cross-talk between the cells.

FIGS. 6C-6D include images of cultures in the OC device that demonstrate the morphology of chondrocytes and HUVEC in co-culture.

FIG. 7 includes images of cultures in the OC device to demonstrate a triculture of the osteoblasts, chondrocytes, and vascular endothelial cells.

FIG. 8 includes a graph that shows the response of the cells to an osteoarthritis stimulator with and without treatment with rapamycin.

FIG. 9 includes a schematic diagram of a computing device (e.g., computing system) that can be used to control the systems and perform the protocols and data analysis as described herein.

FIGS. 10A-10F include graphs that show the fold change of biomolecules in cartilage and bone cultures in the OC device.

FIGS. 11A-11F include data that show the MMP changes in response to the combination of defined cytokines and drug candidates.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology provides an in vitro model of an osteochondral (OC) unit in a device configuration for use in obtaining in vitro OC data. The OC device is designed to use physiologically relevant cells in device chambers that have dimensions that are physiological-like. As such, the width of the chambers can be dimensioned according to measured cartilage and bone portions. The vasculature chamber can also be dimensioned as blood vessels. It is thought that the physiological characteristics of the in vitro OC model device can obtain in vitro data that is relevant to the in vivo data of a real OC region of an organism, such as a human (e.g., real OC data).

The in vitro model of the OC unit can be configured as a 3D microfluidic device (e.g., in vitro OC device) that is configured to be able to provide a real-time visualization and quantification of vasculature cells, bone environment, and cartilage environment that are individually co-cultured in separate chambers with porous interconnections to allow for tissue-tissue crosstalk. The in vitro OC device can be used to differentiate human mesenchymal stem cells (hMSCs) into bone and cartilage lineages, which can be monitored by visualization techniques using cameras (e.g., still images or video). The changes to the cells in the in vitro OC device can then be studied for a model of osteoarthritis (OA), such as by inducing an OA-like inflammation by LPS, by a defined mixture of cytokines, or by introduction of synovial fluid obtained from patients with OA inflammation. Additionally, the in vitro OC device can be used to monitor and examine the vascularized bone-cartilage response to a proposed treatment, such as administration of rapamycin or other potentially therapeutic agent. This allows for screening a number of therapeutic agents for treatment of OA.

The in vitro OC device allows for obtaining in vitro data that can be used to predict physiological conditions and responses to stimuli, such as screening for active agents for treating OA and other diseases or disorders of the joint or OC region. The in vitro OC device is configured as a microfluidic device with different chambers that cooperatively and accurately represent the complex physiology of the OC region. A native OC extra cellular matrix can be used in the in vitro OC device with the osteogenic cells and chondrogenic cells to mimic the OC in an in vivo environment. The different OC regions in the in vitro OC device can be evaluated for viability, sustainability, and functionality as compared to any in vitro, in vivo, or ex vivo OC data from literature or obtained from experiments. That is, the in vitro data from the in vitro OC device can be correlated with in vivo data from a real OC.

The microfluidic in vitro OC device includes an OC architecture with flow channels and a pump system that provides flow rates that yield flow and transport patterns similar to those found in the in vivo OC environment. The in vitro OC device contains: i) a fluidic channel to provide the fluid to the device, which can be devoid of cells or include endothelial cells or epithelial cells (e.g., immortalized, primary or iPSC-derived), which channel is channel 1 herein; ii) a cartilage channel in which chondrocyte cells or other cartilage-related cells (e.g., immortalized, primary or iPSC-derived) are cultured, which channel is channel 2 herein; iii) a bone channel in which osteoblasts or other bone cells (e.g., immortalized, primary or iPSC-derived) are cultured, which channel is channel 3 herein; and iv) a vasculature channel in which vascular endothelial cells (e.g., immortalized, primary or iPSC-derived) are cultured, which channel is channel 4 herein. The channels in the microfluidic network can be considered to be chambers for the purposes of description of regions within the fluidic pathways.

A porous architecture separates the channels/chambers, but allows communication via several, repetitive nanometer to micrometer sized gaps. Barrier integrity of cartilage regions and bone regions can be evaluated by permeability or resistance/impedance. Following exposure/insult, impact to cartilage and bone barrier function can be evaluated and compared to normal conditions with different test agents. As used herein, a channel has a chamber with an inlet and an outlet. The different chambers of channels 1-4 that are described herein can be provided as channels with inlets and outlets. However, either one or both of the cartilage channel or bone channel could be configured without an inlet and/or outlet, but could also be provided as channels with inlets and/or outlets to control media supply or other control properties, as well as introduce the respective cells into the proper culture chambers in the channels.

The in vitro OC device addresses the need for a standardized platform that improves understanding of the OC physiology and agent transport enables prediction of OC drug transport in vivo without actually using a real OC region of a subject. The microfluidics-based in vitro OC device provides physiologically relevant data while enabling real-time morphological, pharmacokinetic and toxicological evaluations. For example, an imaging system, or any other assay system can be operably coupled with the in vitro OC device so as to be able to obtain real time data thereof. Physiological relevance is established by the presence of tissue specific molecules (e.g., hydroxyapatite for bone or aggrecan for cartilage), which can be generated by the cells in the in vitro OC device.

Cell morphology, proliferation and tight junction functionality are all endpoints that can be studied to determine whether or not they are dependent on a suitable ECM for the in vitro OC device. Accordingly, the cells and ECM in each compartment of the in vitro OC device can be configured to mimic the corresponding in vivo structure, such as cartilage, bone, and vasculature. In addition, physiological fluid flow values are used to drive controlled perfusion in the in vitro OC device. Also, quantitative values for biomarkers can be used to validate the in vitro model relevance by comparison of those same biomarkers.

### In Vitro OC Device

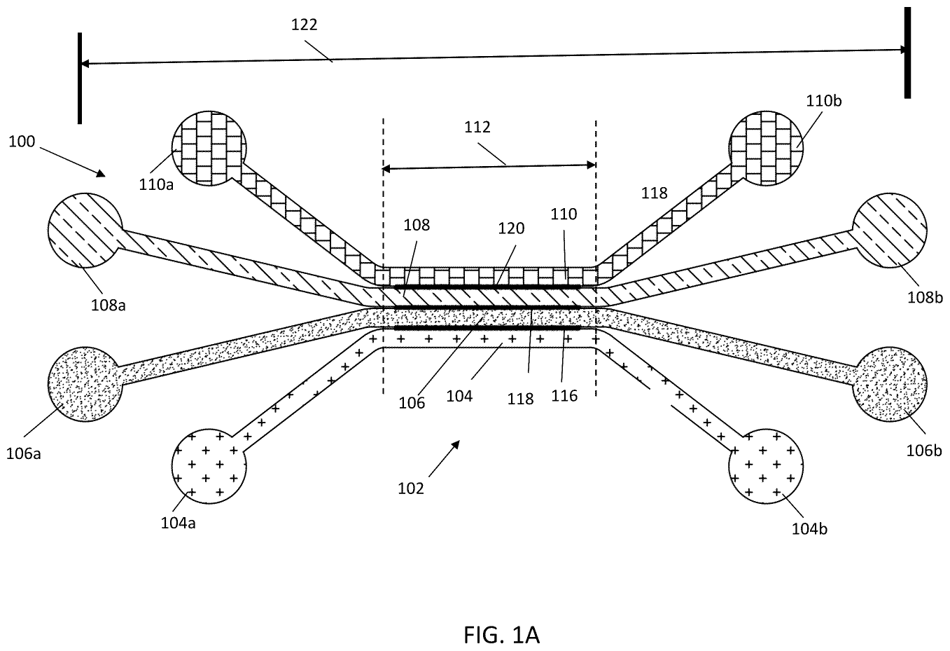

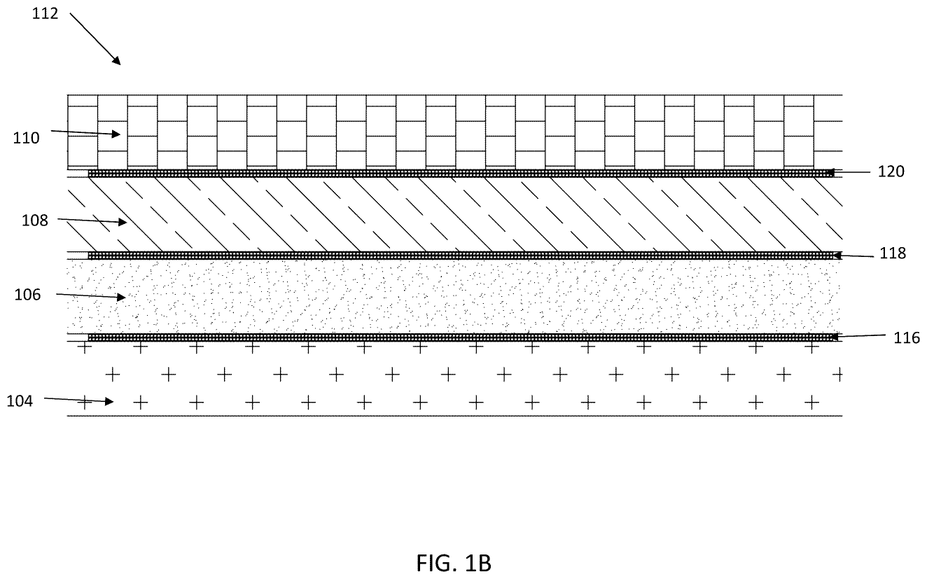

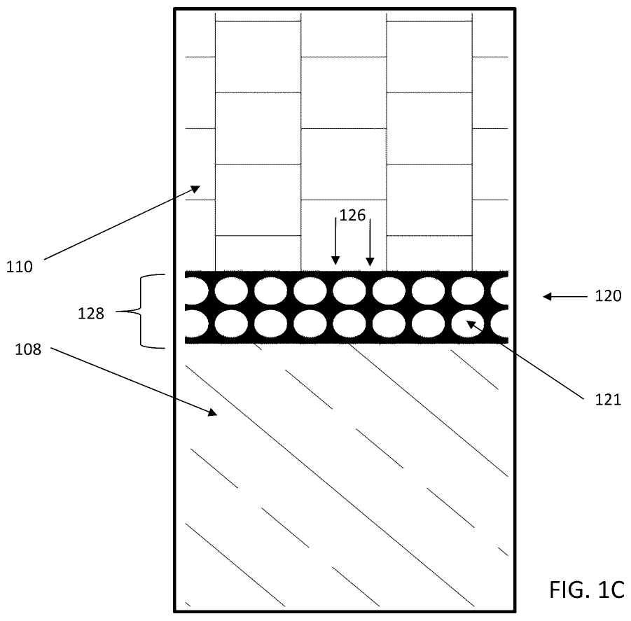

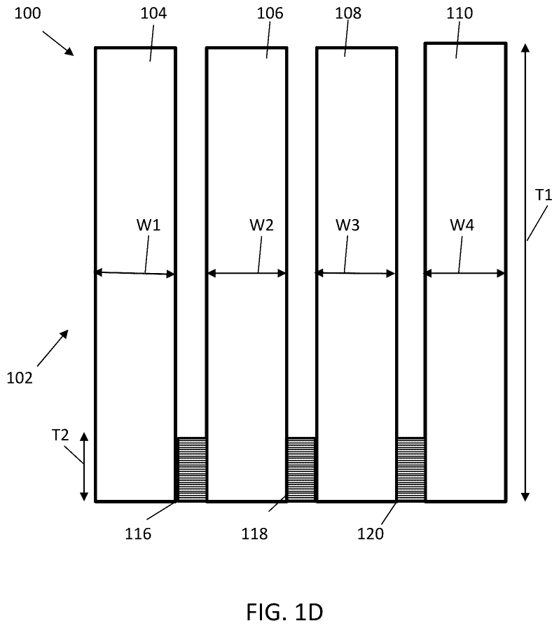

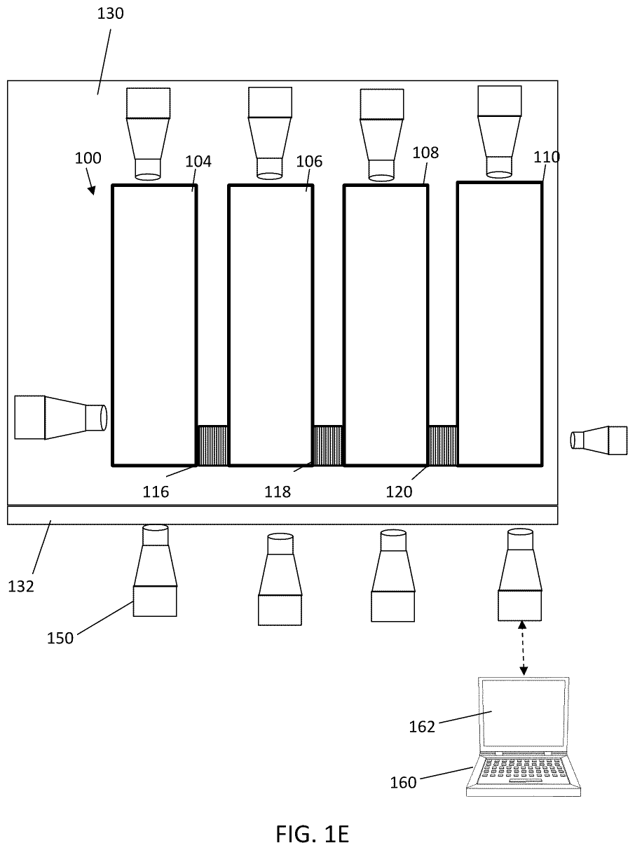

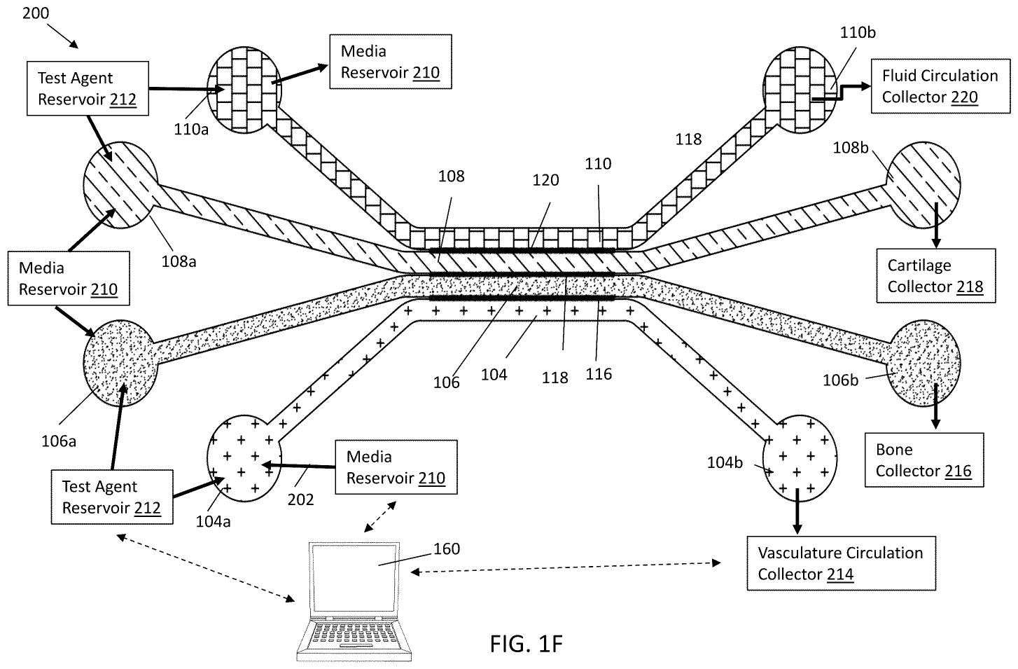

An embodiment of the in vitro OC device 100 is shown in FIGS. 1A-1D. FIG. 1A includes a top view of the in vitro OC device 100 having the microfluidic network 102 that includes the vasculature chamber 104, bone chamber 106, cartilage chamber 108, and fluidic chamber 110. The vasculature chamber 104 is shown to include an inlet 104a and an outlet 104b, with the vascular cell culture region therebetween, with or without extracellular matrix material (e.g., natural or synthetic), or other biological molecules or hydrogels. The vascular cell culture region (e.g., vascular endothelial cells) can be the region between the marker lines for the assay region 112. As shown, the assay region 112 can be about 5 mm in length, but can vary as described herein. The bone chamber 106 is shown to include an inlet 106a and an outlet 106b, with the bone cell culture region therebetween, which is the region between the marker lines for the assay region 112. Accordingly, the bone chamber 106 may include an osteocyte, osteoblast, or osteoclast cell culture with or without extracellular matrix material (e.g., natural or synthetic), or other biological molecules or hydrogels. The cartilage chamber 108 is shown to include an inlet 108a and an outlet 108b, with the cartilage cell culture region therebetween with chondrocytes, chondroblasts, and chondroclasts, which is the region between the marker lines for the assay region 112. The fluidic chamber 110 is shown to include an inlet 110a and an outlet 110b, with or without a fluidic cell culture region therebetween, which is the region between the marker lines for the assay region 112.

The chambers are separated by porous walls, such as a first porous wall 116 separating the vasculature chamber 104 from the bone chamber 106, a second porous wall 118 separating the bone chamber 106 and the cartilage chamber 108, and a third porous wall 120 separating the cartilage chamber 108 and the fluidic chamber 110. The dimension from inlet to outlet for the microfluidic length 122 can be 21.7 mm across, but can vary as described herein. Also, each chamber is shown to have a transport dimension between the porous walls. The transport dimension 124 can be 500 microns across, but can vary as described herein. Notably, a plurality of the in vitro OC devices 100 can be used together in a system, such as in an any series or parallel, or combination thereof. The OC device can be formed into a substrate, which substrate has a dimension of 30.7 mm by 18 mm.

FIG. 1B is an enlarged portion of the assay region 112, which shows more details of the vasculature chamber 104, bone chamber 106, cartilage chamber 108, and fluidic chamber 110. Here, the first porous wall 116 is shown separating the vasculature chamber 104 from the bone chamber 106, a second porous wall 118 separating the bone chamber 106 and the cartilage chamber 108, and a third porous wall 120 separating the cartilage chamber 108 and the fluidic chamber 110.

FIG. 1C shows an enlargement of the third porous wall 120 separating the cartilage chamber 108 and the fluidic chamber 110. However, the figure could as well represent any of the first porous wall 116 or second porous wall 118 between their respective channels. Here, it is shown that the third porous wall 120 includes two rows of posts 121 that function to form the porous wall. FIG. 1C also shows the individual pore channels 126 in the porous walls (e.g., 116, 118, 120) between the posts 121, which can have a width of 3 microns, but can vary as described herein. The pore channels 126 can be separated from each other by wall sections 128, which can have a width of 50 microns, but can vary as described herein. The wall sections 128 may be formed by the rows of posts 121. In some aspects, a single row of posts 121 can be used. Also, the posts can have a polygonal or circular cross-sectional profile.

FIG. 1D shows a side view of a schematic representation of the in vitro OC device 100 of FIGS. 1A-1C. As shown, the vasculature chamber 104 has a width W1 across of 500 microns, the bone chamber 106 has a width W2 of 500 microns, the cartilage chamber 108 has a width W3 of 500 microns, and the fluidic chamber 110 has a width W4 of 500 microns. However, each respective chamber can have a width in a range from about 50 microns to about 3000 microns, from about 100 microns to about 1000 microns, from about 250 microns to about 750 microns, or about 400 microns to about 600 microns.

These chambers can all have a thickness T1 (e.g., height) of 100 microns, or from about 10 microns to about 1000 microns, from about 25 microns to about 750 microns, from about 50 microns to about 500 microns, or from about 75 microns to about 250 microns.

FIG. 1D also shows that the porous walls 116, 118, 120, all have a width of 50 microns and a thickness T2 of 8 microns. The individual posts 121 can have a diameter or width of 20 microns. The pore channel 126 between adjacent posts 121 can have a width of 3 microns. The length of the pore walls 116, 118, and 120 can be 5 microns. Each porous wall includes a plurality of pore channels that have a width that ranges from about 3 microns to about 8 microns and a height that ranges from about 6 microns to about 10 microns. Each pore channel is spaced from about 5 microns to about 75 microns apart from another pore channel. However, these dimensions can be varied as described herein, such as by 1%, 2%, 5%, 10%, 25%, or even 50%.

FIG. 1E shows a substrate 130 having the in vitro OC device 100 formed therein, and a glass slide 132 (or other lid) on a side. The substrate 130 can be PDMS or other biocompatible structural material. The glass slide 132 can be glass or plastic, such as PDMS.

The configuration of the device and use of PDMS (polydimethylsiloxane) or other polymer (e.g., polystyrene, cyclic olefin copolymers, polyethylene terephthalates, etc.) that is optically clear provides the ability for real-time, high content, quantitative imaging of vascular endothelial cells in the vasculature chamber, osteogenic cells (e.g., osteocyte, osteoblast, osteoclast) in the bone chamber 106, chondrogenic cells (e.g., chondrocyte, chondroclast, chondroblast) in the cartilage chamber 108, and the fluidic chamber 110 can include cells (e.g., endothelial, epithelial) or be devoid of cells.

FIG. 1E shows cameras 150 that can be placed outside of the glass slide 132, outside the substrate 130, or embedded in the substrate 130 (e.g., in etched regions). The cameras 150 can be communicatively coupled with a computing system 160 configured as a controller and to receive optical data from the cameras 150. The cameras 150 can take still images or videos of the different chambers of the in vitro OC device. While a number of cameras 150 are shown in an arrangement, the placement and number of cameras can be modified in order to obtain the desired data, where more or fewer cameras can be used. As such, the entire device or select regions of interest can be imaged with the imaging system. The computing system 160 can be communicatively coupled with the cameras 150 by wire, optics, or wireless communication networks, represented as the dashed lines. The computing system 160 can include a display 162 for visually showing the images obtained from the cameras 150, or computed data thereof.

The invention comprises a device, which can be referred to as an in vitro OC device, lab-on-a-chip OC device, or OC-on-a-chip device, designed for the purpose of analyzing a biological structure of an OC region. This device is composed of several interconnected systems and components.

The synovial chamber is designed to mimic a physiological fluid circulation that can provide fluids to tissues. The synovial chamber can be connected to an inlet and an outlet so that fluid with or without test agents can be input and flowed so as to be capable of passing through the pores into an adjacent chamber, which is the cartilage chamber. The synovial chamber has one or more pumps (e.g., microfluidic pumps or micropumps), and optionally one or more reservoirs, such as a cell culture media reservoir, test agent reservoir, positive control reservoir, or negative control reservoir.

The cartilage chamber is designed to mimic the cartilage layer in a real space adjacent with bone. This cartilage chamber is also connected to the same fluidic network and devices as the previous component. The cartilage chamber can receive fluid through a porous wall from the synovial chamber. The output from this cartilage chamber is fluid that is directed towards the bone chamber through another porous wall.

The bone chamber is designed to mimic the bone region, which can include hard materials. This space is also connected to the network and devices as the vasculature circulation system, such as with one or more pumps (e.g., microfluidic pumps or micropumps), and optionally one or more reservoirs, such as a cell culture media reservoir, test agent reservoir, positive control reservoir, or negative control reservoir. The output from this space is fluid that is directed towards the next component, the vasculature circulation.

Another device component is the vasculature system, with is configured as a microvascular network or microchannel network. This vasculature system is designed to mimic the blood flow in a real osteochondral region. It is connected to a microfluidic network with one or more pumps (e.g., microfluidic pumps or micropumps), and optionally one or more reservoirs, such as a cell culture media reservoir, test agent reservoir, positive control reservoir, or negative control reservoir. the vasculature system can receive input from the bone chamber. The output from this system is fluid that can be collected.

In some embodiments, the system is configured for flow to be from the fluidic chamber through the cartilage chamber, then through the bone chamber, and then to the vasculature chamber.

In some embodiments, the system is configured for flow to be from the vasculature chamber through the bone chamber, then cartilage chamber to the fluidic chamber.

FIG. 1F shows an exemplary system 200 that includes the in vitro OC device 100. As shown, a fluidic network including a media reservoir 210 feeding any one of the chambers, which can include one or more pumps to facilitate fluid flow. As such, each line such as 202 can include a fluidic pathway and a pump with one or more valves. However, each chamber may have its own media reservoir, which can include a specialized media configured for the cell culture therein, such as growth factor, or other biomolecules. Also, a test agent reservoir 212 is shown to feed into any one or more of the chambers, which can be selectively via valves and/or pumps in the lines 202. The test agent reservoir 212 can include test agents to be used in the methods described herein on monitoring the cartilage and bone, and monitoring the transport properties thereof. Any number of test agent reservoirs 212 can be used for any number of test agents. The test agents can be any type of test agent, such as those described herein.

FIG. 1F also shows the outlet of each chamber coupled to a particular collector, which can include one or more pumps (e.g., each fluid line 202 can include a pump and associate valves) to move the fluid. The pumps can be used to precisely control the fluid flow through the channels and chambers. The vasculature chamber 104 is connected to a vasculature circulation collector 214 adapted to collect the fluid from the outlet 104b. The bone chamber 106 is connected to a bone collector 216 adapted to collect the fluid from the outlet 106b. The cartilage chamber 108 is connected to a cartilage collector 218 adapted to collect the fluid from the outlet 108b. The fluid circulation chamber 110 is connected to a fluid circulation collector 220 adapted to collect the fluid from the outlet 110b. Various valves or other fluidic network components can be included, such as heaters, coolers, or the like. The components of the system 200 can be controlled by the computing system 160.

The microfluidic in vitro OC devices can be fabricated using standard PDMS soft-lithography techniques as known. CAD drawings of the microfluidic OC device geometry can be generated to create SU-8 silicon molds, and device architecture is realized by casting with PDMS. Inlet and outlet ports are punched into the PDMS mold and then the structure is then bonded to clean glass slides to form the final microfluidic device prototypes. Alternatively, the glass slides can be configured as lids or covers for the microfluidic network.

In some embodiments, the in vitro OC device can be prepared by forming the body with a polymeric material, such as a transparent polymer. Examples can include polystyrene, cyclic olefin copolymer (COC), polyethylene terephthalates, and the like. The polymer body can be prepared by hot embossing the transparent polymer, and then bonding the bodies by using heat or a UV curable adhesive.

### In Vitro OC Device Coatings and Cultures

The in vitro OC devices are primed with sterile phosphate buffer solution (PBS) by injection into each of the channels. All chambers of the OC device may be coated with various proteins or substrates, creating an extracellular matrix to support the attachment and growth of cells on inner surfaces of the chambers. Example substrates include, but are not limited to, fibronectin, collagen and lyophilized extracellular matrix (ECM). The methods for coating various surfaces (e.g., glass, plastic) with proteins and other substrates are well known in the field. Cells, such as the chamber-specific cells may be cultured on the coated inner surfaces of the relevant chambers to study species transport with respect to the bone and cartilage chambers.

Additionally, end point assays such as those for viability, phenotypic protein expression, metabolic activity, gene expression, and the like can be assessed on- or off-device. For example, fluidic samples can be obtained at the outlet of each chamber throughout an assay and/or cell samples can be obtained after an assay for biomarker profile analysis. Along with visualization, and sample analysis assay can be used with samples obtained from the different compartments. The biochemical analysis along with the visual analysis can be useful in modeling the in vitro system.

### Example In Vitro OC Device Embodiments

In some embodiments, any of the chamber or channels can have a width of about 500 microns, or a range from about 200 microns to about 1000 microns, about 250 microns to about 900 microns, about 300 microns to about 800 microns, about 400 microns to about 600 microns. The porous walls can each have a width of about 50 microns, or a range from about 20 microns to about 100 microns, about 25 microns to about 90 microns, about 30 microns to about 85 microns, or about 40 microns to about 60 microns. The height of the device, or the height of any of the foregoing chambers or features can vary from about 10 microns to about 1000 microns, from about 50 microns to about 500 microns, or about 100 microns to about 200 microns, or about 150 microns. These values may be varied, such as +/−1%, 5%, 10%, 25%, 50%, 75%, or 100% thereof.

In some embodiments, any of the chambers or channels can have a length of about 14 mm, or a range from about 10 mm to about 25 mm, from about 13 mm to about 21 mm, or about 15 mm to about 20 mm. The porous walls can each have a length of about 5200 microns, or a range from about 1000 microns to about 10000 microns, about 2000 microns to 8000 microns, or about 4000 microns to about 6000 microns. In some aspects, these values can be modified, such as by +/−1%, 5%, 10%, 25%, 50%, 75%, or 100% thereof.

In some embodiments, each porous wall can include pore channels therethrough that are 5 microns wide that are spaced 50 microns apart. The width of each pore channel may range from about 1 micron to about 10 microns, about 2 microns to about 8 microns, or about 3 microns to about 7 microns. The heights of each pore channel can ranges from about 6 microns to about 10 micron, or about 8 microns. The spacing of the channels can be from about 5 microns to about 100 microns, about 10 microns to about 90 microns, or about 25 microns to about 75 microns. These values may be modified, such as by +/−1%, 5%, 10%, 25%, 50%, 75%, or 100% thereof.

In some embodiments, the in vitro OC device can include a viewing window into at least one of the chambers or channels of the system. However, each of these systems or chambers can be configured for optical viewing. These systems or chambers can have a clear lid or top, or any other surface, to allow for optical viewing of the content thereof. This can allow for labeling and other colorimetric techniques for monitoring the cultures as well as flow of components and test agents throughout the system.

In some embodiments, an assay device can be configured with the in vitro OC device. The assay device can include a substrate; and a plurality of the microfluidic in vitro OC devices in or on the substrate. The plurality of microfluidic in vitro OC devices can be in parallel or sequential, or combinations thereof.

In some embodiments, a microfluidic in vitro OC system can include: the microfluidic in vitro OC device of one of the embodiments; and at least one pump configured for pumping fluid through the microfluidic in vitro OC device.

In some embodiments, a microfluidic in vitro OC system can include: the microfluidic in vitro OC device of one of the embodiments; at least one camera device configured to be positioned to image at least one of the chambers or channels; and a computing system operably coupled with the at least one camera device to receive image data. In some aspects, the computing system is configured to obtain data from the at least one camera device and determine at least one trans-channel transport barrier property of the microfluidic in vitro OC device or at least one trans-channel transport property of a test agent, wherein the trans-channel transport barrier property is a measurement of inhibition of transport of an agent across the particular channel or combination of bone and cartilage chamber and the trans-chamber transport property of a test agent is a measurement of traversal of the test agent across the respective chamber(s). In some aspects, the computing system includes one or more computer-readable media storing instructions that when executed cause operations that determine the at least one trans-channel transport barrier property of the microfluidic in vitro OC device or the at least one trans-channel transport property of a test agent.

In some embodiments, the in vitro OC device can be used in a method of studying the osteochondral region. The method can include providing the in vitro OC device of one of the embodiments. The device can be used for determining a condition of the in vitro OC device at a first time point. The condition can be any physiological condition of the cells, fluid flow, test analyte, or other feature. Then, the method can include determining the condition of the in vitro OC device at a subsequent time point. That is, the same feature can be measured at a later time, such as after exposure to a certain condition or test analyte. Then, a computing system can be used for determining a change in the condition of the in vitro OC device.

In some embodiments, the method of studying the OC can include: imaging at least one of the channels through a viewing window of the device; viewing images in real time of the at least one channel through a display screen of a computing system; or measuring transport across the bone channel and/or cartilage channel of at least one of nutrients, xenobiotics, small molecules, lipids, liposomes, polymers, particles, toxins, and antibodies from data in the images. That is, the image is analyzed to determine the transport properties.

In some embodiments, the method of studying the OC can include: providing the microfluidic in vitro OC device of one of the embodiments; providing a test agent to an input chamber selected from at least one of the channels; and monitoring trans-channel transport of the test agent across the bone and/or cartilage channels.

In some embodiments, a method of studying transport of the test agents can include at least one of: determining an amount of test agent crossing the cartilage chamber and bone chamber and comparing the amount of test agent that crossed the cartilage chamber and bone chamber with the administered amount of the test agent introduced into the microfluidic in vitro OC device (e.g., into the fluidic chamber); sampling the vasculature chamber for the test agent and quantifying the transport of the test agent across the cartilage and bone chambers into the vasculature chamber; or sampling the fluidic chamber for the test agent and quantifying the transport of the test agent across the cartilage and bone chambers into the fluidic chamber. Herein, the assay can be a test agent from the synovial chamber to the vasculature circulation chamber, or from the vasculature circulation chamber to the synovial chamber, and both are considered as embodiments in a protocol when either embodiment is discussed.

In some embodiments, the methods can include evaluating OC barrier function of the OC barrier chamber by: injecting a plurality of different test agents having a plurality of different sizes into one of the fluidic chamber or vasculature chamber; imaging the in vitro OC device; analyzing images of the in vitro OC device to identify the plurality of different test agents; and determining a size of test agent or size range of test agent of the plurality of test agents located in the different chambers. Each chamber can be analyzed visually.

In some embodiments, the methods can include determining at least one of: a size of test agent or size range of test agents capable of transporting from the vasculature circulation chamber across the bone and cartilage chambers into the synovial chamber; a lipophilicity of test agent or lipophilicity range of test agents capable of transporting into different chambers; or a physiological charge of test agent or charge range capable of transporting into a different chamber or across the bone and cartilage chambers. In another aspect, the transport can be from the synovial chamber into the cartilage, bone, and vasculature chambers.

In some embodiments, the methods can include evaluating permeability of the in vitro OC device by: injecting one or more test agents into the fluidic or vasculature chamber; imaging the microfluidic in vitro OC device; analyzing images of the microfluidic in vitro OC device to identify locations of the test agent at defined time points, and optionally determine amounts of each test agent in each chamber at the time points; and determining a permeability of the in vitro OC device for the one or more test agents.

In some embodiments, the methods can include determining a permeability index as a ratio of optical intensity measurements of the vasculature circulation chamber with the synovial chamber.

In some embodiments, the methods can include evaluating whether the test agent modifies permeability or structural integrity or morphology of the cells in a chamber by: determining an initial value of a first property of the chamber; introducing the test agent into the microfluidic in vitro OC device; determining a subsequent value of the first property of the chamber; and determining a difference between the initial value and the subsequent value of the first property of the chamber. The value can be related to one or more of structural integrity or morphology of the chamber or other feature, as well as features of the test agent. This can be done for the bone, cartilage, or vasculature chambers.

In some embodiments, the methods can include determining a health consequence of the test agent modulating the bone and/or cartilage chamber by correlating the difference between the initial value and the subsequent value and a phenotypic state, which phenotypic state may or may not be a disease state or disorder state.

In some embodiments, the methods include determining whether or not a test agent changes inflammation of the cells in at least one of the synovial chamber, cartilage chamber, bone chamber, or vasculature circulation chamber. Such a change in inflammation can be in response to addition of a test agent. Also, the cells may be treated to induce inflammation, and then a test agent can be used to see whether or not the test agent can treat inflammation. The inflammation can be monitored visually with a camera and the morphology or other changes can be monitored visually to detect changes in inflammation, whether causing inflammation or treating inflammation. The most frequently used inflammatory biomarkers include acute-phase proteins, essentially CRP, serum amyloid A, fibrinogen and procalcitonin, and cytokines, predominantly TNFα, interleukins 1β, 6, 8, 10 and 12 and their receptors and IFNγ, which can be measured herein to determine changes thereof in response to a test agent or other condition. Any of the methods recited herein can be performed as described, and the added steps of monitoring inflammation can also be performed simultaneously.

In some embodiments, changes in biomolecules can be monitored in response to the test agent. That is, the transcriptome products, proteome products, metabolites, or other biomolecules of the cells described herein may be monitored for changes upon change of a condition, such as introduction of a test agent. The monitoring of the biomolecules can be added to any of the methods described herein, which can provide valuable information when changes are detected. Also, changes in cell morphology can be monitored in response to test agents, which can be done visually.

In some embodiments, the cells can be monitored for viability in response to any of the methods described herein. That is, any of these methods can be performed, and the cell viability can be determined at one or more time points. The cell viability can be determined visually, such as with viability test agents that show whether or not the cells are alive. For example, cells that are alive do not take in dye, so the presence of a dye visually within a cell can indicate poor cell viability. Cell viability studies are well known in the art.

In some embodiments, the method of studying transport of test agents can include determining a lipophilicity of test agent or lipophilicity range of test agent capable of transporting across the bone chamber and/or cartilage chamber, or into either respective chamber. As such, a panel of different agents with a gradient of lipophilicity can be used and monitored in order to create a lipophilicity profile for test agents, which can provide a lipophilicity range of the in vitro device. The amount of lipophilicity of a certain substance type can be assessed to determine whether such lipophilicity modulates the transport thereof or other test agents in the in vitro OC device.

In some embodiments, the method of studying transport of test agents can include determining a physiological charge of test agent or range thereof capable of transporting across the bone chamber and/or cartilage chamber. Accordingly, the different charges of different test agents can be monitored across a panel for transport in the in vitro OC device. The pH may also be varied to monitor the modulation that charges can have on the transport phenomena.

In some embodiments, the method of studying transport of test agents can include evaluating permeability of the in vitro OC device. The permeability may compromise the transport data that can be determined to make sure the data is physiologically relevant to in vivo OC parameters. The permeability can be assessed by: injecting test agent into the system (e.g., fluidic channel or vasculature channel); imaging the in vitro OC device; analyzing images of the in vitro OC device to identify locations of the test agent and optionally determine amounts of test agent in each location; and determining a permeability of the in vitro OC device for the test agent. The test agent may or may not be labeled with a visible label, such as fluorescent label. The method can also include evaluating permeability of the in vitro OC device by: injecting test agent into the fluidic circulation system; imaging the vasculature circulation system; analyzing images of the device to identify the test agent; and determining a permeability of the in vitro OC device for the test agent. The evaluation of permeability of the in vitro OC device can also be performed by: injecting test agent into the vasculature circulation system; imaging the device; analyzing images of the device to identify the test agent; and determining a permeability of the in vitro OC device for the test agent.

In some embodiments, the method of studying transport of test agents can include determining a permeability index. The permeability index can be defined as a ratio of a sum of optical intensity measurements of one or more region of interests (ROIs) bone and cartilage chambers. This ratio between the optical intensity measurements of the fluidic and vasculature circulation systems can be used to define the permeability.

In some embodiments, these methods can be performed by assaying the cells, fluids, reagents, and cell products (e.g., metabolites of reagents) while in the in vitro OC device. That is, the in vitro OC device can be configured to allow for monitoring the cells, fluids, reagents, and cell products while still in the in vitro OC device. Such assays performed directly on the chip can be referred to as “on-chip” analyses. An example of on chip analysis can include images, videos, temperature, pressure, flow rate, colorimetric changes (e.g., reagent and cell products are different colors), microscopy (e.g., phase contrast microscopy or confocal microscopy), or any other experiment done on the in vitro OC device and cells, fluids, reagents, and cell products therein. Examples of on-chip assays can include MTT assay, visual morphologic assay, fluorescent assay, BrdU assay, ATP assay, immunofluorescence, live cell imaging, high content screening, and others.

In some embodiments, the cells, fluids, reagents, and cell products may be obtained from the in vitro OC device and assayed off of the in vitro OC device, which is different from assays that are on-chip. For example, these materials may be retrieved from the in vitro OC device and introduced into an assay system that is off-chip (e.g., performed elsewhere from the in vitro OC device. Examples of off-chip assays can include live/dead assay, LDH assay, mass spectroscopy, ELISA, Western Blotting, RT-qPCR, RNA-seq, flow cytometry, high throughput screening, and others.

### Differentiation

In some embodiments, a method of differentiating cells is performed. The method can include differentiating cells into osteogenic cells and chondrogenic cells. The differentiated osteogenic cells and chondrogenic cells can then be cultured in adjacent culture chambers separated by porous walls. The differentiation method can include providing the in vitro OC device of one of the embodiments. The method can include introducing first human mesenchymal stem cell into the cartilage chamber. Then, the method can include introducing a chondrogenic differentiation medium into the cartilage chamber with the first human mesenchymal stem cells. The first human mesenchymal stem cells can be incubated with the chondrogenic differentiation medium sufficiently to differentiate into at least one of chondrocyte cells, chondroblast cells, and/or chondroclast cells. However, chondrocyte cells are demonstrated to be obtained with chondrogenic differentiation medium, as shown herein. A second batch of human mesenchymal stem cells can be incubated with osteogenic differentiation medium sufficiently to differentiate into at least one of osteoblast cells, osteoclast cells, and/or osteocyte cells, where osteoblasts are demonstrated herein. The differentiated at least one of osteoblast cells, osteoclast cells, and/or osteocyte cells can be obtained or introduced into the bone chamber. Also, the vascular endothelial cells can be introduced into the vasculature chamber, which can be before, during or after introduction of the osteoblasts into the bone chamber.

In some embodiments, the differentiation method can include incubating the second human mesenchymal stem cells with osteogenic differentiation medium sufficiently to differentiate into at least one of osteoblast cells, osteoclast cells, and/or osteocyte cells in a separate cell culture chamber. This can be a T-flask (e.g., such as a T-75 mL flask). The differentiated at least one of osteoblast cells, osteoclast cells, and/or osteocyte cells can be obtained from the separate cell culture chamber.

In some embodiments, a removable divider can be included between the cartilage chamber and the bone chamber. The divider can be physically removed by opening the lid and removing the divider. The divider can fit in the porous wall around the posts, or therebetween, to provide a barrier. Also, the barrier can be a biodegradable barrier that degrades over time. For example, a biodegradable barrier can be placed between the cartilage chamber and the bone chamber, which can then be degraded over the next 1, 3, 5, 7, 12, 14, 20, or 24 days. The thickness and consistency of biodegradable barrier can be tailored by the polymers used to control the degradation rate. This configuration allows the osteogenic cells to be differentiated from the hMSCs at the same time as the chondrogenic cells are differentiated from the hMSCs. For example, hMSCs can be flowed into the bone chamber and cartilage chamber that are separated, and the osteogenic differentiation medium can be introduced into the bone chamber while cartilage differentiation medium can be supplied into the cartilage chamber. This can allow for co-differentiation in the device. The removable or degradable divider can be removed as desired or as degraded.

In some embodiments, the differentiation method can include incubating the vascular endothelial cells with growth medium in a second separate cell culture chamber. Then, the vascular endothelial cells can be obtained from the second separate cell culture chamber. This allows for the endothelial cells to be grown in a T-flask (e.g., T-75) for culturing. Also, the first human mesenchymal stem cells are introduced into the cartilage chamber before differentiation, and the differentiation in the OC device occurs before the osteogenic or vascular endothelial cells are introduced. In some aspects, the method can include introducing growth medium to the first human mesenchymal stem cells before introducing the chondrogenic differentiation medium. In some aspects, the differentiated at least one of osteoblast cells, osteoclast cells, and/or osteocyte cells are introduced into the bone channel. In some aspects, the vascular endothelial cells are introduced into the vasculature circulation channel. However, a removable divider (e.g., biodegradable) can be used between the bone chamber and vasculature chamber so that the differentiation into bone cells can occur adjacent to the vascular endothelial cells, and the divider can be removed when appropriate and the bone cells are differentiated. However, it may be possible to induce differentiation of the cells into bone cells in the bone chamber while the vascular cells are present in the vasculature chamber without a barrier between these respective chambers.

In some embodiments, the hMSCs are placed into the cartilage chamber and bone chamber with or without a removable divider. However, the differentiation method can be without a removable divider when controlling the flow rates. For example, the flow rates of the cartilage and bone chambers can be set to be about the same. The flow rates to the fluid chamber and vascular chamber can be off or set low so that flow in the cartilage region diverts into the fluid chamber away from the bone chamber, and the flow in the bone region diverts into the vasculature chamber away from the cartilage chamber. This can be used for co-differentiating into the chondrogenic cells in the cartilage chamber with the chondrogenic differentiation medium at the same time as co-differentiating into the osteogenic cells in the bone chamber with the osteogenic differentiation medium. The fluid flows can be controlled to minimize fluid flow between the bone and cartilage chambers.

In some embodiments, the differentiation method can include introducing the first human mesenchymal stem cells and growth media into the cartilage chamber at an initial time point. Then, the method can include introducing the chondrogenic differentiation medium into the fluidic circulation channel and/or cartilage chamber from 1 to 20 days after the initial time point. The differentiated at least one of osteoblast cells, osteoclast cells, and/or osteocyte cells can be introduced into the bone chamber from 1-20 days after the initial time point. The vascular endothelial cells can be introduced into the vasculature circulation chamber from 1-20 days after the initial time point. The growth medium can be supplied to the synovial chamber and/or cartilage chamber after cessation of chondrogenic differentiation medium. The growth medium can be supplied to the bone chamber and/or vasculature chamber after introduction of the differentiated at least one of osteoblast cells, osteoclast cells, and/or osteocyte cells into the bone chamber and/or introduction of the vascular endothelial cells into the vasculature circulation chamber.

For example, hMSCs can be thawed and seeded into a T-flask with growth medium. Once 80% confluent, the hMSCs can be seeded into the OD device. On day 1, hMSCs are perfused into channel 2, which is the cartilage chamber. Also, the hMSCs are supplemented with growth media into channel 1 (e.g., synovial chamber) on day 1 (e.g., 10 microliters per hour).

On days 2-16, the channels 1 (fluidic), 3 (bone), and 4 (vasculature) are rinsed with growth medium or flushing fluid (e.g., saline). Then, channel 1 is perfused with chondrogenic differentiation medium (e.g., 10 microliters per hour). All perfusing can be between 1 microliter per hour to 20 microliters per hour. Also additional hMSCs can be introduced to osteogenic differentiation medium in a separate T-flask. Additionally, HUVEC cells (human umbilical vein endothelial cells (HUVECs) are cells derived from the endothelium of veins from the umbilical cord) are seeded with growth medium (e.g., EGM-2).

Also, on days 2-16, the channels 1, 3, and 4 can be rinsed with growth medium. The differentiated osteoblasts can be perfused into the bone chamber. Also, the HUVEC cells can be perfused into the vasculature circulation chamber.

On days 17-18, a growth medium (e.g., EGM-2) can be supplied to channel 1 at 10 microliters per hour. Also, channel 3 can receive growth medium (e.g., EGM-2) at 5 microliters per hour. Additionally, channel 4 can receive growth medium (e.g., EGM-2) at 3 microliters per hour. These flow rates can be continuous, or for periodic intervals, such as for 5 minutes per every 1, 2, 3, or 4 hours.

On day 18-24, an assay is performed (e.g., initiated) with the OC device having the differentiated chondrocytes, differentiated osteoblasts, and vascular endothelial cells.

Additionally, the methods described herein can be performed with cells other than hMSCs or cells differentiated from hMSCs. In some embodiments, the cells used in the in vitro OC device can be primary cells. Primary cells are cells that are isolated directly from living tissues or organs and cultured for use in experiments or research. Unlike established cell lines, which have undergone transformation to enable continuous growth in culture, primary cells have a finite lifespan in culture and retain many of the characteristics of the original tissue from which they were derived. This makes them more representative of in vivo conditions compared to cell lines. However, immortalized and other types of cells could also be used.

### EXAMPLES

### Materials

Dulbecco's Modified Eagle Medium (DMEM) and Fluorobrite cell culture medium, fetal bovine serum (FBS), antibiotic-antimycotic (penicillin, streptomycin, and fungizone, PSF), insulin-transferrin-selenium (ITS), TrypLE Express, 2.0 μm blue fluorescent FluoSpheres™ Carboxylate-Modified Microspheres were purchased from ThermoFisher Scientific. Red fluorescent 2.0 μm red polymer microspheres were obtained from Duke Scientific. Dragon Green fluorescent tagged, 4.19 μm polystyrene microspheres were obtained from Bangs Laboratories, Inc. Human recombinant transforming growth factor β3 (TGF-β3) was obtained from Peprotech, Inc. 1α,25-Dihydroxyvitamin D3 was obtained from Enzo Life Sciences. Human Umbilical Vein Endothelial Cells (HUVECs) and the Endothelial Cell Growth Medium-2 (EGM-2) BulletKit were obtained from Lonza. All other materials were obtained from Sigma Aldrich.

### Device Fabrication

The in vitro OC device was prepared as a 4-channel microfluidic device using AutoCAD LT (Autodesk) simultaneously culture three cellular layers of the OC unit: vasculature, bone, and cartilage (from the bottom to the top). The fourth channel (channel 1) was included above the cartilage layer to permit feeding of the hydrogel that fills the cartilage channel (channel 2) as the cartilage channel will not permit direct perfusion, thus mimicking the absence of vascular network in native cartilage and providing a synovial space. The porous barrier between each channel consists of 20 μm pillars spaced 3 μm apart. The spaces between the pillars allows cell-cell communication and molecular diffusion across the barrier while preventing cell migration (FIGS. 1A-1C).

Prototypes were fabricated using standard polydimethylsiloxane (PDMS) soft-lithography techniques. Briefly, SU-8 silicon molds were fabricated and a Sylgard 184 PDMS kit was utilized for fabrication of the microfluidic devices, similar to previous work [31]. Briefly, a 10:1 mixture of the two-part Sylgard 184 kit components was prepared and poured onto the mold. The mold was placed inside a vacuum desiccator and degassed for 1 hour. The silicone mold was then placed in an oven and cured for 4 hours at 65° C. The cast PDMS was removed and cut into individual devices. Inlet and outlet ports were punched in the PDMS and the devices were plasma bonded to clean glass slides to form the final microfluidic devices. Further, FIG. 1A shows the manufactured microfluidic device with the four channels.

### Barrier Integrity and Size Validation

The in vitro OC device was validated to monitor the barrier size and integrity by perfusing the device with fluorescent dextran and fluorescent particles. To validate complete PDMS attachment throughout the device, including the barrier pillars, the system perfused the device with 10 kDa fluorescein-dextran (FITC-dextran). In addition, the inter-pillar gap size was validated by perfusing channels with one of three fluorescent bound polystyrene particles: 2 μm red fluorescent particles, 2 μm blue fluorescent particles, and 4 μm green fluorescent particles. During perfusion, the device was imaged on a Nikon T1-2 inverted microscope with attached fluorescence source.

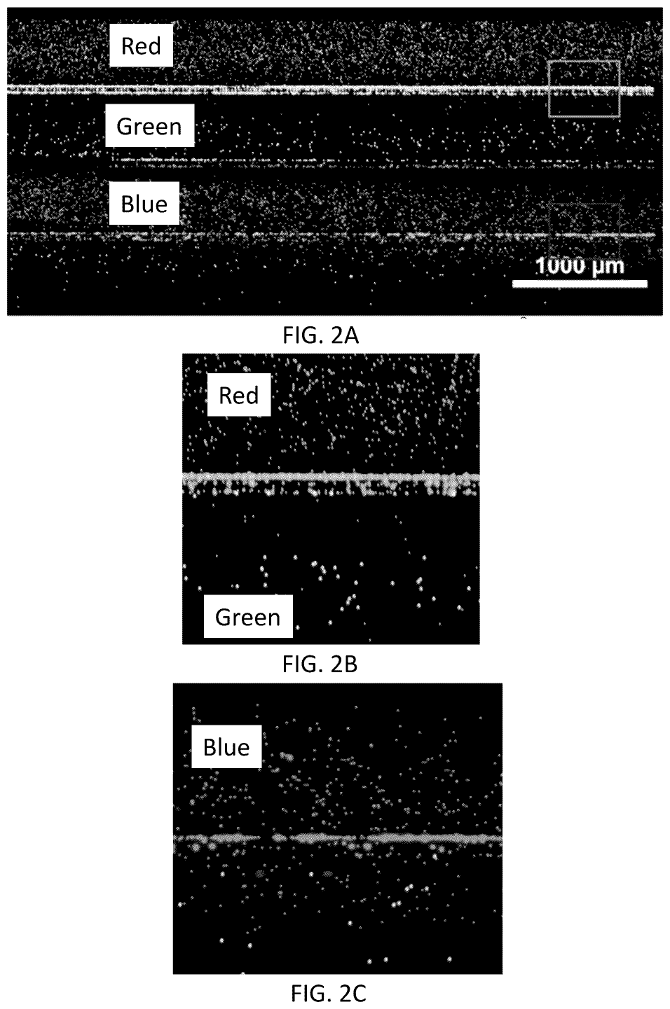

The shape, dimensions, and barrier integrity of the OC device were verified by perfusing the channels with 10 kDA FITC-dextran. When perfused with FITC, the fluid tightness of the device is completely intact, both channels and barriers, as indicated by the restriction of the dye to those areas. FIG. 2A shows that the manufactured barriers match the specified design gap of 3 μm. As shown, while 2 μm fluorescently tagged polystyrene particles (red and blue) were able to pass from one channel to another, 4 μm FITC tagged polystyrene particles were maintained in their respective channels and were unable to cross the barriers (FIG. 2A-C). This validates the microfluidic architecture of the OC device. The data provides validation of PDMS-glass bonding and lack of obstructions in channels. FIG. 2A shows the validation of pore size and barrier integrity with fluorescent polystyrene beads (blue and red in outer channels are 2 μm, green are 4 μm in inner channel). FIG. 2B is a zoom in of the red 2 μm particles passing through the barrier. FIG. 2C is a zoom in of blue 2 μm particles passing through the barrier. However, the larger 4 μm particles do not pass through the barrier.

### Cell Culture

The hMSCs were extracted from femoral heads of patients undergoing total joint arthroplasty). Before the experiments, cells were maintained from thaw in growth medium (GM: Gibco™ FluoroBrite™ DMEM, FBS (10% v/v), PSF (2% v/v), sodium pyruvate (1% v/v) and GlutaMAX™ (1% v/v)), at 37° C., 5% CO₂ until they reached ˜80% confluency. HUVEC were maintained in identical, routine culture conditions in complete EGM-2 media. For all experiments, hMSCs and HUVEC were used at passage 3 or 4.

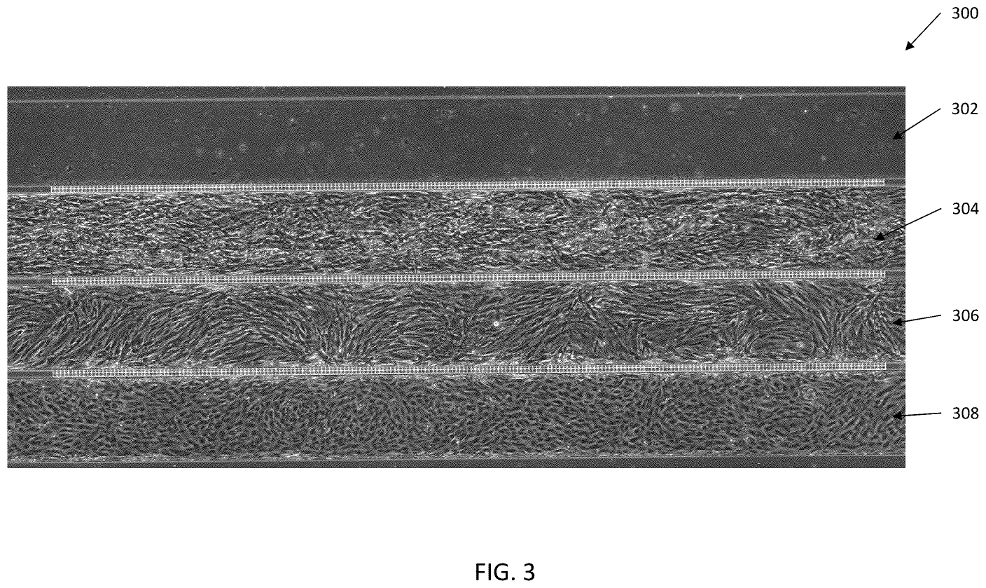

FIG. 3 includes a micrograph showing the OC device 300 having channels comprised of synovial fluid 302, differentiated chondrocytes cell culture 304, differentiated osteoblasts cell culture 306, and vasculature endothelial cell culture 308. One of more cell type is maintained in their respective channel and mimic the layers of the osteochondral unit. However, each channel can be predominantly the particular cell types for the anatomy of the OC region being mimicked.

### Chondrogenic Monoculture

The hMSCs derived chondrocyte monoculture was established by hMSC differentiation in the microfluidic device. The hMSCs at a concentration of 108 cells/mL were seeded with a syringe pump at a flow rate of 5 μL/min into channel 2 of a device incubated for 1 hour at 37° C., 5% CO₂ with 200 μg/mL fibronectin from human placenta (hFN) and then incubated with 1% (w/v) gelatin for an additional hour to create an ECM coating [32]. Then, the inlet and outlet of the channel were clamped, and the devices were incubated for 1 hour before tubing was connected to channel 1, adjacent to the channel 2 (where cells were seeded), and growth medium (GM) was perfused at a constant rate of 10 μL/hr for 48 hours with the device in the incubator. After 48 hours, channels 1, 3, and 4 were flushed with chondrogenic differentiation medium (CM, consisting of GM supplemented with 10 μg/mL ITS, 0.1 μM dexamethasone, 40 μg/mL L-Proline, 50 μg/mL L-ascorbic acid 2-phosphate, and 10 ng/mL TGF-β3). Then, channel 1 was perfused with CM at 10 μL/hr. Two different differentiation time points were evaluated: 14 days and 28 days. Media was perfused through channel 1 to reduce shear stress on the cells in channel 2. Indeed, shear stress alone has been shown to decrease chondrogenesis of hMSCs and direct them instead towards a myogenic phenotype [33]. Moreover, initial experiments demonstrated that shear caused loss of cells during direct flow over the chondrocytes.

As a positive control, hMSCs were seeded onto a 6-well plate at passage four at a concentration of 300,000 cells per well and cultured for 2 days with GM. Then, GM was changed to CM and cells were cultured for 28 days (to ensure complete differentiation for comparison).

### Osteogenic Monoculture

The hMSCs derived osteoblast monoculture was established by hMSC differentiation in the microfluidic device. Cells at a concentration of 1×10⁷ cells/mL were seeded via syringe pump at a flow rate of 5 μL/min into channel 3 of a device coated with 200 μg/mL hFN and 1% gelatin +0.1% nano-hydroxyapatite (nanoHA) using GM. After seeding, the device was perfused with GM via syringe pump, directly over the cells, at a flow rate of 5 μL/min. After two days, the device was rinsed (channels 1, 2, and 4) with osteogenic differentiation medium (OM, consisting of GM supplemented with 0.1 mM ascorbic acid, 10 mM β-glycerophosphate, 0.1 μM dexamethasone and 10 nM 1,25-dihydroxy vitamin D₃) and OM was perfused directly over the cells at a flow rate of 5 μL/hr at 37° C. and 5% CO₂. Some devices were maintained on GM without the rinse and subsequent perfusion with OM as a control. Two different differentiation time points were evaluated: 14 and 28 days.

As a positive control, hMSCs were seeded onto a 6-well plate at passage four at a concentration of 300,000 cells per well and cultured for 2 days. Then, GM was changed to OM and the system was cultured for 28 days.

### Establishing Osteoblast and Chondrocyte Monocultures

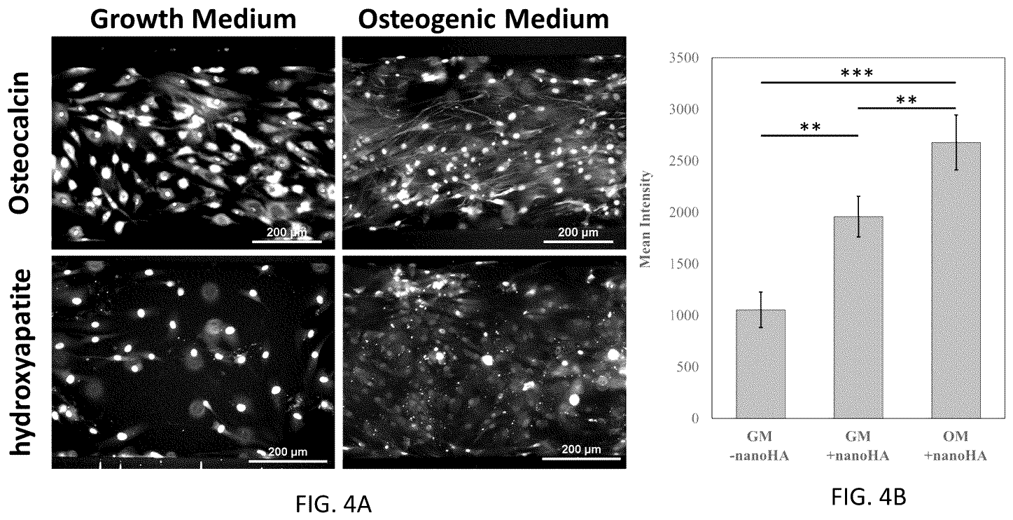

The capability of hMSCs to differentiate toward chondral and osseous linages in the microfluidic device was established by seeding and then differentiating the cells towards both lineages separately in the devices as described herein. Strong staining for osteocalcin for the hMSCs that underwent bone differentiation via exposure to OM over two weeks in the device was observed, compared to hMSCs cultured with GM over the same period in the device, as shown in FIGS. 4A and 4B. Interestingly, 14 days of culture with OM in the microfluidic OC device is sufficient to obtain a mature osseous differentiation and no differences were observed between 14 and 28 days of culture. Osteocalcin appeared to migrate from the nucleus (in the control) to form filamentous structures after differentiation (device supplied with OM). To confirm the osteogenic commitment of cells, devices were stained with the OsteoImage mineralization assay to stain hydroxyapatite in hMSC seeded devices before and after differentiation (FIGS. 4A-4B), and with and without nanoHA in the device coating. It was confirmed that the levels of hydroxyapatite in the device channels rose significantly in the presence of OM, whether or not nanoHA was incorporated into the coating, indicating hMSCs underwent osteogenic differentiation, thus leading to hydroxyapatite deposition by differentiated osteoblasts. The graph in FIG. 4B shows the presence of hydroxyapatite, and shows that the presence is increased when hydroxyapatite is added to the system. Additional hydroxyapatite is present with just the differentiation medium, indicating that the hMSCs have differentiated into osteoblasts and are producing hydroxyapatite.

Regarding the cartilage monoculture, after two weeks of chondrogenesis, hMSCs contracted into a cartilage-like structure (FIG. 5) adjacent to the bone channel (channel 3) and stained strongly for aggrecan. Similar to bone differentiation, 14 days of culturing in the microfluidic device is sufficient to obtain a mature chondral differentiation and no substantial differences were observed between 14 and 28 days of culture.

Notably, FIG. 5 shows the data of Aggrecan and Collagen II for day 3 and Day 20. As shown, significant Aggrecan and Collagen II are found at day 20.

### Co-Culture System

To establish hMSC derived osteoblast and vascular coculture (osteogenic coculture) or hMSC derived chondrocyte and vascular coculture (chondrogenic coculture), the respective monoculture, the osteogenic culture or chondrogenic culture was firstly established as described herein. Then, channel 4 was coated with 200 μg/mL hFN to ensure a complete basement membrane, and the devices were rinsed with EGM-2 medium. Then, HUVECs were seeded into channel 4 at a density of 5×10⁷ cells/mL. The devices were then maintained by continuous flow of EGM-2 medium at a flow rate of 5 μL/min on channel 3 and intermittent flow of EGM-2 medium on channel 4 (3 μL/min for 5 min every 4 hours) for the osteogenic coculture, and continuous flow of EGM-2 medium at a flow rate of 10 μL/min on channel 1 and intermittent flow of EGM-2 medium on channel 4 (3 μL/min for 5 min every 4 hours) for the chondrogenic coculture. The cells in the OC device were cultured with this set up for two days.

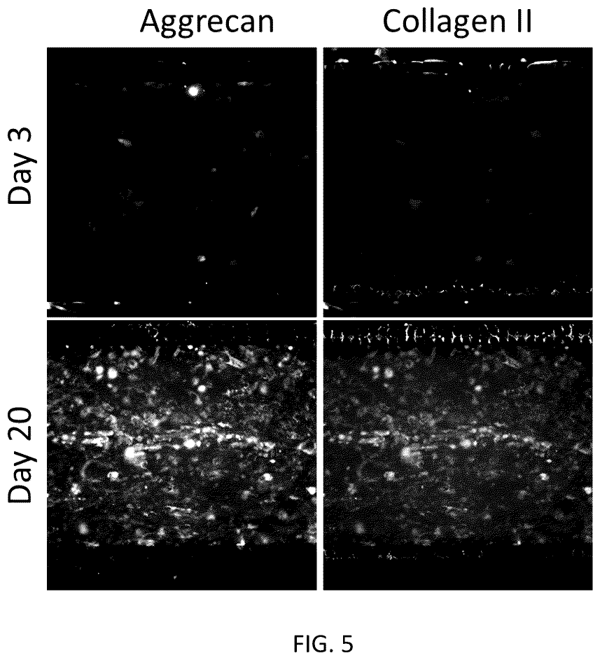

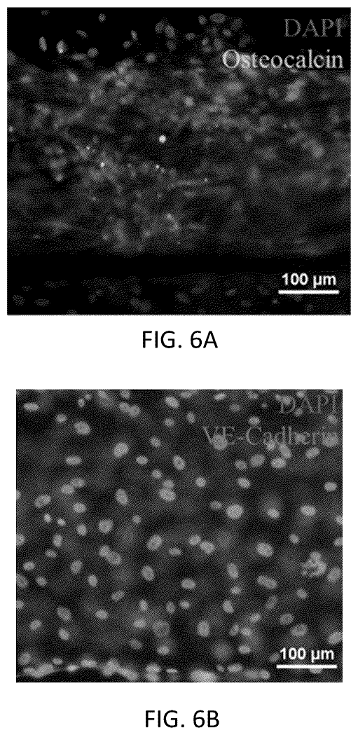

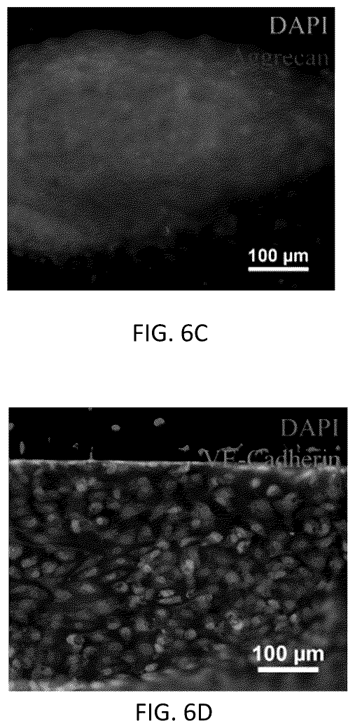

Accordingly, co-cultures of hMSC derived osteoblasts or hMSC derived chondrocytes were established with HUVEC as a first step towards the desired tri-culture device, by establishing the mono-culture and then seeding HUVEC into the 4 channel after initial differentiation. It was determined that all of the cells could be cultured with EGM-2 medium without detrimental effects to the osteoblast or chondrocyte components, similar to our previous work [39,40]. As shown in FIGS. 6A-6D, the system using the device successfully obtained and maintained a co-culture of HUVEC and either osteoblasts or chondrocytes in EGM-2, post differentiation. FIGS. 6A-6B demonstrate the morphology of osteoblast and HUVEC that are side-by-side, which permits cross-talk between the cells. FIG. 6A-6B show immunostaining for osteocalcin and VE-Cadherin demonstrating that the cells are confined to their specific channels. Similarly, FIGS. 6C-6D demonstrate the morphology of chondrocytes and HUVEC in co-culture, where FIGS. 6C-6D demonstrate positive staining for aggrecan and VE-Cadherin.

### Triculture Systems

To establish the complete triculture, a chondrogenic monoculture was established culturing the cells for 14 days in the microfluidic device as previously described in 2.5. Simultaneously, hMSCs underwent osteogenic differentiation in a T75 culture plate. The protocol can differentiate hMSC derived osteoblasts and chondrocytes separately to prevent hMSCs to be cultured with mixed differentiation signals due to the close spacing and open pore hierarchy of the microfluidic devices that facilitate diffusion between channels, especially since OM might further differentiate hMSC derived chondrocytes into osteoblasts [34-36]. It was determined to not to attempt osteogenic differentiation followed by chondrogenic differentiation inside the device in a serial manner because it would have significantly increased the total time required to establish the model. After two weeks of differentiation in plate, the hMSC derived osteoblasts were seeded concurrently with HUVEC at a concentration of 1×10⁷ cells/mL and 5×10⁷ cells/mL respectively and cultured for an additional 2 days with EGM-2 medium, in all channels.

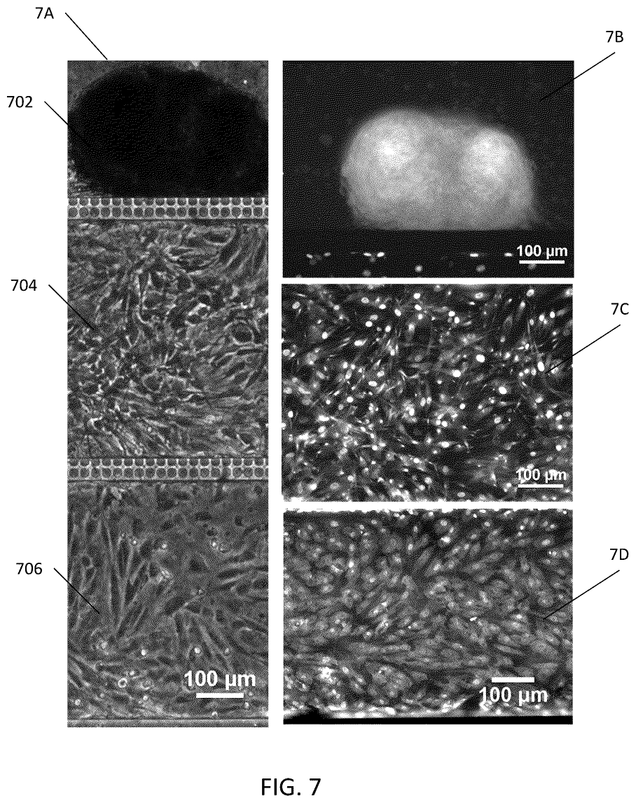

To establish the complete tri-culture, co-differentiated hMSCs into osteoblasts and chondrocytes were obtained. Since subjecting chondrocytes to osteogenic medium changes their morphology [34-36], the protocol seeded the devices with hMSCs and began chondrogenic differentiation for two weeks, while simultaneously differentiating hMSCs into osteoblasts in T75 cell culture plates. Then the protocol seeded osteoblasts and HUVEC simultaneously. For the following two days, HUVECs were subjected to intermittent flow at a rate of 3μL/min for 5 min every 4 hours. FIG. 7 includes panel 7A shows the morphology of chondrocytes 702, osteoblasts 704, and HUVEC 706 in side by side channels. Similarly, panels 7B, 7C, and 7D shows differentiation by immunocytochemistry and location of the three cell types confined into the adjacent channels.

### Immunostaining

Differentiation of hMSCs into either the osteogenic or chondrogenic lineage were validated by immunostaining for osteocalcin and aggrecan/collagen II, respectively. Differentiation was validated in monoculture, co-culture, and tri-culture microfluidic devices, as well as the well plate controls. Further, in the co-culture and tri-culture devices, HUVEC were stained for an endothelial specific marker, such as vascular endothelial (VE)-cadherin.

The detailed steps are provided herein for the tri-culture, of which the monoculture and coculture staining protocols are identical, except for the exclusion of stains for non-present cell types.

Microfluidic devices were fixed with 3.7% formaldehyde for 20 minutes at room temperature and rinsed with phosphate-buffered saline (PBS). Then samples were blocked overnight with 10% bovine serum albumin (BSA) and 0.1% triton-x 100 in PBS followed by incubation overnight at 4° C. The following day, the triculture device was stained with primary antibodies: osteocalcin, a calcium binding protein found in bone, (R&D Systems MAB1419, 8 μg/mL) for hMSC derived osteoblasts, and aggrecan, the major extracellular proteoglycan of cartilage, (R&D Systems AAF1220, 5 μg/mL) for hMSC derived chondrocytes. The primary antibodies were mixed together to create a master mix in 1% BSA and 0.1% Triton X-100 and perfused in all channels. The devices were then incubated overnight at 4° C. with the primary antibodies. The next day, the devices were rinsed with PBS and perfused with secondary antibodies (Jackson Immunoresearch Alexa Fluor 488 Donkey anti-mouse, Cat. No. 715545150, and Alexa Fluor 647 Donkey anti-goat, Cat. No. 705605003) diluted in 1% BSA and 0.1% triton-x 100 in PBS and incubated overnight at 4° C. To complete the immunostaining, the devices were rinsed with PBS and perfused with the final primary antibody, VE-Cadherin, an endothelial and brain specific protein involved in cell-cell connection (BioLegend 348512, 5 μg/mL, tagged with Alexa Fluor 598) for HUVEC specific staining, and incubated for 1 hour in the dark at room temperature. Finally, devices were rinsed and perfused with NucBlue fixed cell stain (ThermoFisher R37606 2 drops to 960 μL of PBS) before a final wash with PBS. Stained devices were imaged on a Nikon T1-2 inverted microscope with attached fluorescence source.

### OsteoImage Mineralization Assay

For the osteogenic monoculture, the protocol utilized the OsteoImage Mineralization Assay (Lonza), which binds to the hydroxyapatite deposited by osteoblasts, to demonstrate generation and deposition of hydroxyapatite. This mineralization assay provided a secondary validation of osteogenic differentiation. To validate hydroxyapatite production beyond the hydroxyapatite provided in the coating, the protocol stained several device conditions: 1) hMSCs seeded into channel 3 of devices coated with gelatin alone and supplied with GM (negative control), 2) hMSCs seeded into channel 3 of devices coated with gelatin plus nanoHA and supplied with GM, and 3) devices coated with gelatin plus nanoHA and supplied with OM. Staining was conducted according to the manufacturer's protocol. Briefly, after fixing the devices with 3.7% paraformaldehyde, they were washed by perfusion with PBS followed by a 1:10 dilution of the kit's wash buffer (a 2X wash was accomplished by doubling the perfusion volume in devices). After rinsing, the staining reagent at a 1:100 dilution was perfused into all channels and permitted to incubate for 30 minutes at room temperature, protected from light. The devices were then washed 3X with wash buffer and imaged on an inverted microscope using a FITC filter cube and LED light source.

### Inflammatory Assay

In the tri-culture device, OA-like cues were supplied to observe i) oxidative stress induced by LPS conditioning, and ii) the reduction of the oxidative stress in the simultaneous presence of LPS and the anti-inflammatory drug Rapamycin (RAP). Thus, three conditions were investigated: no insult (control,-LPS-RAP), insult (+LPS−RAP), and insult plus treatment (+LPS+RAP). For the insult, the medium was exchanged in established devices with 100 ng/mL LPS perfused into channel 3 to maximize inflammation across the device by supplying LPS in the closest proximity to both bone and vasculature. For the insult plus treatment, medium was exchanged in established devices with 100 ng/mL LPS perfused into channel 3, and 20 ng/mL RAP perfused into channel 4 to simulate the supply of drug from the vasculature, as RAP is orally administered and so it is expected to diffuse from the vasculature into the affected area. The devices for all cases were then incubated for 4 hours without flow.

Resultant oxidative stress was measured by image processing after four hours by the CellROX™ Orange Reactive Oxygen Species Detection Kit (Thermo Fisher), a fluorometric reactive oxygen species (ROS) assay, according to the manufacture's protocol. Briefly, CellROX Orange was prepared in EGM-2 medium at a concentration of 5 μM and perfused into all channels. Devices were then incubated for 30 min at 37° C. followed by a 3X rinse with PBS and immediate imaging using an inverted microscope. The same microscope was used to capture images for each experiment and exposure and other microscope settings were kept constant to allow quantitative comparisons between micrographs. Quantitative measurement of relative ROS was conducted by image analysis via ImageJ and Matlab®. Briefly, for each condition (i.e., i)-LPS-RAP; ii)+LPS-RAP; iii)+LPS+RAP) images were split into three regions of interest (ROIs), representing the three channel of the devices (vasculature, bone, cartilage), that were separately analyzed. Then the images were converted in an 8-bit image, and the brightness was adjusted within same range in all the images. Then, images were binarized using the same threshold, assuming the pixels above the threshold as information (e.g., ROS), and the pixels below it as background. Finally, for each image the pixels representing the information were summed and normalized by the total pixel number of the image, thus obtaining a normalized sum of the intensity of the image that represent the level of oxidative stress of each device region at each condition. Three different devices were analyzed for each condition (n=3).

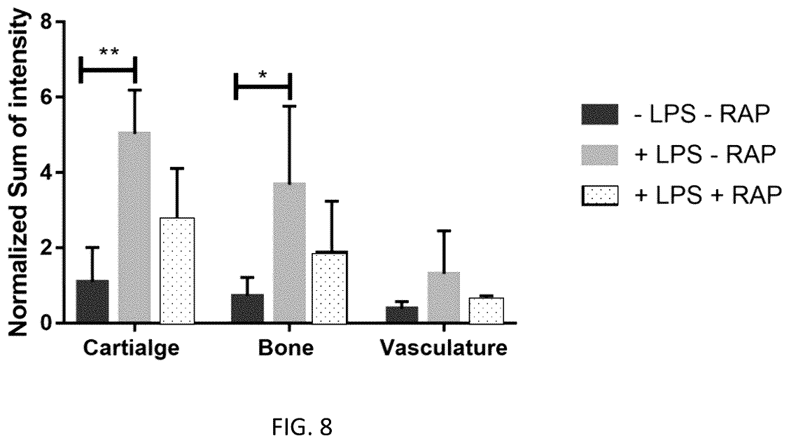

Evidence exists that a bacterial infection of the joints may be one OA etiology [18]. Accordingly, the protocol was performed to examine oxidative stress across the completed model in response to LPS, a common bacterial inflammatory signal. The protocol examined the reduction of oxidative stress when the inflamed model was treated with RAP, a well-known anti-inflammatory compound, thus showing the ability of the OC device platform to correctly induce the OA related stress of tissue, and the beneficial effect of recognized drugs. As shown in FIG. 8, the inclusion of LPS in the device increases the oxidative stress of the cells in all the compartment (channels) with a statistically significant variation in the chondral and bone channels. Interestingly, the inclusion of rapamycin reduced inflammation across all three compartments, bringing the system back to a non-inflammatory state. Indeed, no statistical difference appear between the control (darkest bar, no LPS and no RAP), the initial state (light being with LPS and no RAP), and the final state (dot texture, is positive for LPS and positive for RAP).

### Statistical Analysis

Statistical analysis was performed by one-way analysis of variance (ANOVA) combined with a Levene's test for homogeneity of variances and a Tukey HSD post-hoc analysis. Data obtained by image analysis were analyzed via 2-way ANOVA with Tukey HSD post hoc analysis to pinpoint any statistical difference among data.

qPCR

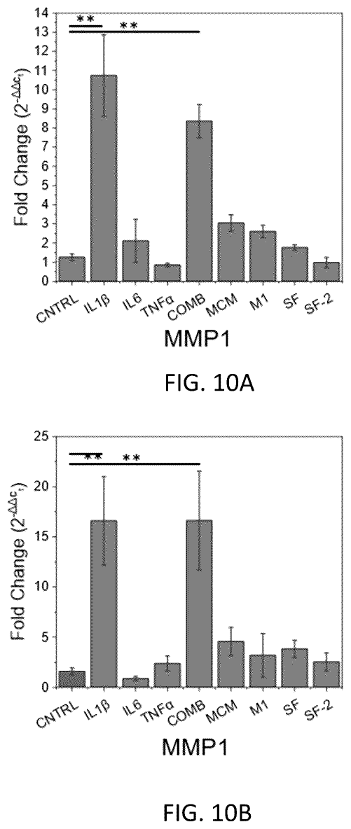

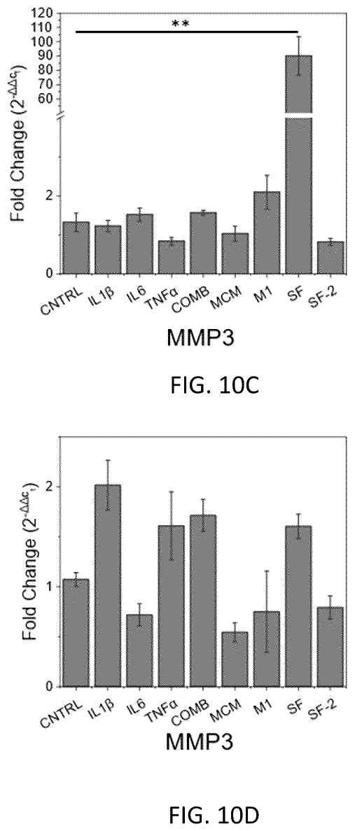

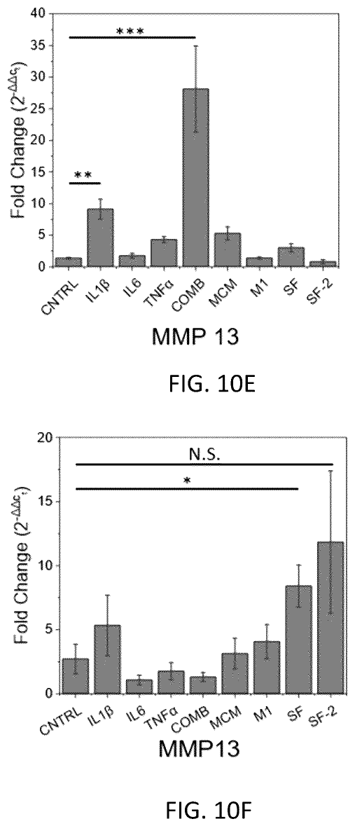

The qPCR data from extracted cells was obtained from the cells that can be individually extracted on a per-channel basis so that tissues can be analyzed separately. Data in FIGS. 10A-10F shows changes in MMPs (e.g., catabolic genes related to OA) in response to exposure for 24 hours of: 1) defined inflammatory cytokines (e.g., IL1-β, IL6, and TNFα) separately; 2) combined at 100 ng/ml; 3) macrophage conditioned medium (MCM); 4) inflammatory macrophages (M1) perfused into the synovial channel; 5) Synovial fluid set 1; and 6) Synovial fluid set 2. Statistical significance determined by Kruskal-Wallis non-parametric test followed by Dunn's post hoc. * p<0.05, ** p<0.01, and *** p<0.001. FIGS. 10A, 10C, and 10E show bone. FIGS. 10B, 10D, and 10F show cartilage.

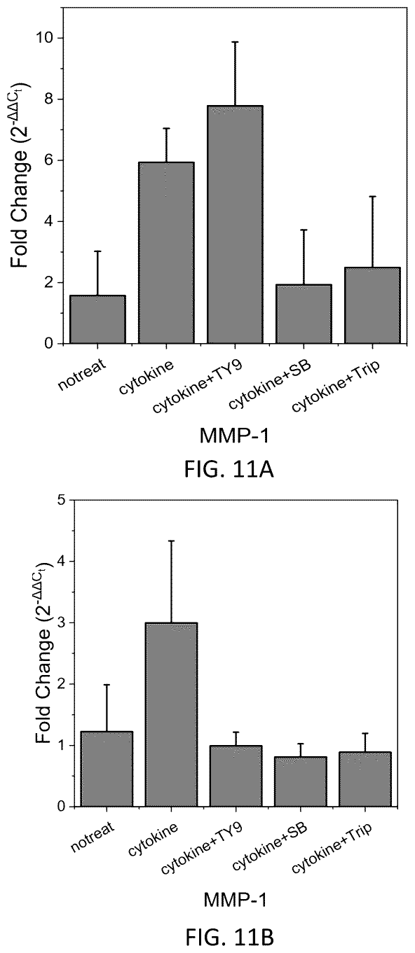

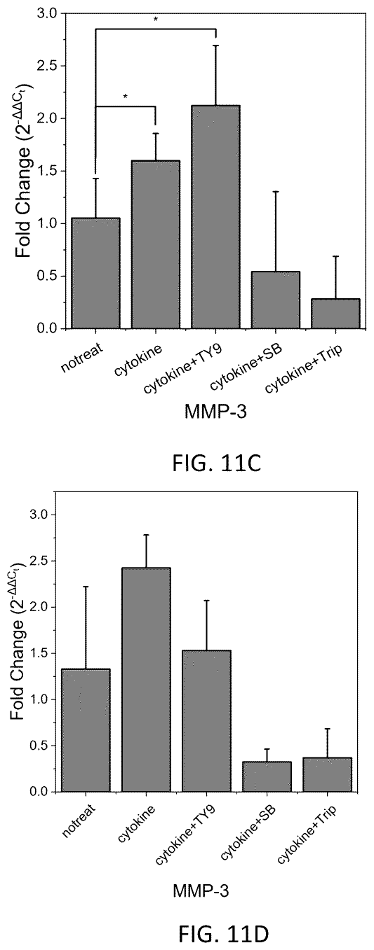

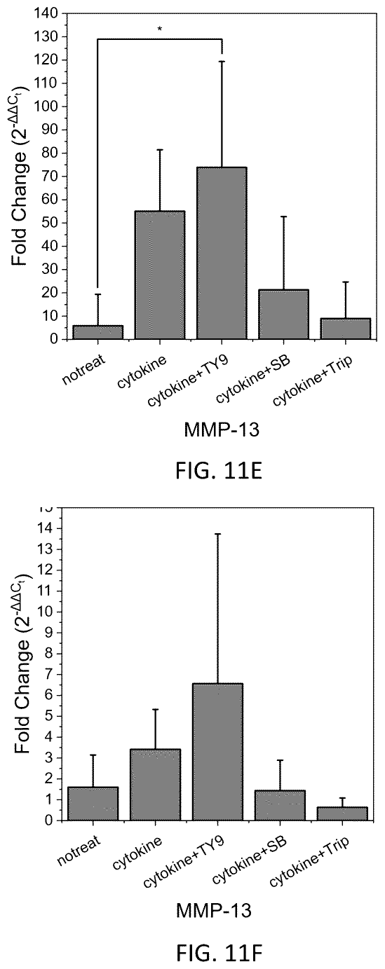

FIGS. 11A-11F include data that show the MMP changes in response to the combination of defined cytokines and a drug candidate. Devices were exposed to a combination of defined inflammatory cytokines for 24 hours. After 24 hours, the devices were exposed to either fresh medium without cytokines, or to Tyrphostin-9 (TY9), a candidate compound with potential anti-inflammatory effects under consideration as a palliative for osteoarthritis (OA). As can be seen, there is an increase in fold change from the no treatment, the cytokine only, or the cytokine and Tyrphostin-9. This shows the inflammatory condition is improved with addition of the Tyrphostin-9 over no treatment and cytokine alone. Thus, the device can be used to screen for drugs to treat OA.

This shows that any potential drug can be screened with the OC device as described herein for determining whether or not the potential drug can be a candidate for treating OA.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system, such as control of the in vitro OC device and system thereof or obtaining data or analysis of data from the in vitro OC device. As such, the computing system can include a memory device that has the computer-executable instructions for performing the methods. The computer-executable instructions can be part of a computer program product that includes one or more algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor. The computer-readable instructions can include instructions for operating the in vitro OC device and associated system (e.g., pumps, cameras, analytical equipment), or instructions for analyzing data from the in vitro OC device.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

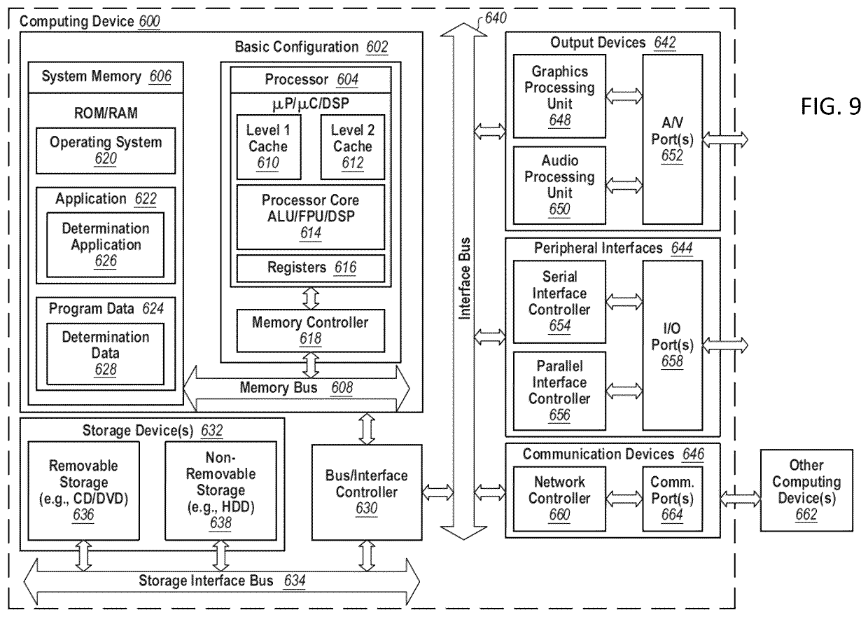

FIG. 9 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method comprising computer-implemented steps as described herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.
ADDIN ZOTERO_BIBL {“uncited”: [ ], “omitted”: [ ], “custom”: [ ]}CSL_BIBLIOGRAPHY [1] H. Lin, T. P. Lozito, P. G. Alexander, R. Gottardi, R. S. Tuan, Stem Cell-Based Microphysiological Osteochondral System to Model Tissue Response to Interleukin-1B, Mol. Pharm. 11 (2014) 2203-2212.
[2] A. D. Woolf, B. Pfleger, Burden of major musculoskeletal conditions, Bull. World Health Organ. 81 (2003) 646-656.
[3] S. Safiri, A.-A. Kolahi, E. Smith, C. Hill, D. Bettampadi, M. A. Mansournia, D. Hoy, A. Ashrafi-Asgarabad, M. Sepidarkish, A. Almasi-Hashiani, Global, regional and national burden of osteoarthritis 1990-2017: a systematic analysis of the Global Burden of Disease Study 2017, Ann. Rheum. Dis. 79 (2020) 819-828.
[4] Osteoarthritis (OA), Cent. Dis. Control Prev. (2020). cdc.gov/arthritis/basics/osteoarthritis.htm (accessed Nov. 30, 2020).
[5] P. G. Alexander, J. A. McCarron, M. J. Levine, G. M. Melvin, P. J. Murray, P. A. Manner, R. S. Tuan, An in vivo lapine model for impact-induced injury and osteoarthritic degeneration of articular cartilage, Cartilage. 3 (2012) 323-333.
[6] A. C. Bay-Jensen, E. Slagboom, P. Chen-An, P. Alexandersen, P. Qvist, C. Christiansen, I. Meulenbelt, M. A. Karsdal, Role of hormones in cartilage and joint metabolism: understanding an unhealthy metabolic phenotype in osteoarthritis, Menopause. 20 (2013) 578-586.
[7] F. Djouad, L. Rackwitz, Y. Song, S. Janjanin, R. S. Tuan, ERK1/2 activation induced by inflammatory cytokines compromises effective host tissue integration of engineered cartilage, Tissue Eng. Part A. 15 (2009) 2825-2835.
[8] M. B. Goldring, S. R. Goldring, Articular cartilage and subchondral bone in the pathogenesis of osteoarthritis, Ann. N. Y. Acad. Sci. 1192 (2010) 230-237.
[9] D. Chen, J. Shen, W. Zhao, T. Wang, L. Han, J. L. Hamilton, H.-J. Im, Osteoarthritis: toward a comprehensive understanding of pathological mechanism, Bone Res. 5 (2017) 1-13.
[10] V. B. Kraus, G. McDaniel, J. L. Huebner, T. Stabler, C. Pieper, R. E. Coleman, N. A. Petry, P. S. Low, J. Shen, P. Mitchell, Direct in vivo evidence of activated macrophages in human osteoarthritis, Osteoarthritis Cartilage. 21 (2013) S42.
[11] P. G. Alexander, R. Gottardi, H. Lin, T. P. Lozito, R. S. Tuan, Three-dimensional osteogenic and chondrogenic systems to model osteochondral physiology and degenerative joint diseases, Exp. Biol. Med. 239 (2014) 1080-1095.
[12] S. I. M. Lepage, N. Robson, H. Gilmore, O. Davis, A. Hooper, S. St. John, V. Kamesan, P. Gelis, D. Carvajal, M. Hurtig, T. G. Koch, Beyond Cartilage Repair: The Role of the Osteochondral Unit in Joint Health and Disease, Tissue Eng. Part B Rev. 25 (2019) 114-125.
[13] C. Carulli, M. Innocenti, M. L. Brandi, Bone vascularization in normal and disease conditions, Front. Endocrinol. 4 (2013) 106.
[14] D. M. Findlay, J. S. Kuliwaba, Bone-cartilage crosstalk: a conversation for understanding osteoarthritis, Bone Res. 4 (2016) 1-12.
[15] S. R. Goldring, M. B. Goldring, Changes in the osteochondral unit during osteoarthritis: structure, function and cartilage-bone crosstalk, Nat. Rev. Rheumatol. 12 (2016) 632-644.
[16] A. Courties, J. Sellam, Osteoarthritis and type 2 diabetes mellitus: What are the links?, Diabetes Res. Clin. Pract. 122 (2016) 198-206.
[17] Q. I. Zhuo, W. Yang, J. Chen, Y. Wang, Metabolic syndrome meets osteoarthritis, Nat. Rev. Rheumatol. 8 (2012) 729-737.
[18] Z. Huang, V. B. Kraus, Does lipopolysaccharide-mediated inflammation have a role in OA?, Nat. Rev. Rheumatol. 12 (2016) 123-129.
[19] R. Ossendorff, S. Grad, M. J. Stoddart, M. Alini, H. Schmal, N. Südkamp, G. M. Salzmann, Autologous chondrocyte implantation in osteoarthritic surroundings: TNFα and its inhibition by adalimumab in a knee-specific bioreactor, Am. J. Sports Med. 46 (2018) 431-440.
[20] C. I. Johnson, D. J. Argyle, D. N. Clements, In vitro models for the study of osteoarthritis, Vet. J. 209 (2016) 40-49.
[21] S. Nasi, A. So, C. Combes, M. Daudon, N. Busso, Interleukin-6 and chondrocyte mineralisation act in tandem to promote experimental osteoarthritis, Ann. Rheum. Dis. 75 (2016) 1372-1379.
[22] Y. Ouyang, W. Wang, B. Tu, Y. Zhu, C. Fan, Y. Li, Overexpression of SOX9 alleviates the progression of human osteoarthritis in vitro and in vivo, Drug Des. Devel. Ther. 13 (2019) 2833-2842.
[23] J. M. Chun, A. Y. Lee, J. Y. Nam, M. Y. Lee, M. S. Choe, K. S. Lim, C. Kim, J.-S. Kim, Protective effects of Phlomis umbrosa extract on a monosodium iodoacetate-induced osteoarthritis model and prediction of molecular mechanisms using transcriptomics, Phytomedicine Int. J. Phytother. Phytopharm. 81 (2021) 153429.
[24] P. J. Cope, K. Ourradi, Y. Li, M. Sharif, Models of osteoarthritis: the good, the bad and the promising, Osteoarthritis Cartilage. 27 (2019) 230-239.
[25] K. H. Benam, S. Dauth, B. Hassell, A. Herland, A. Jain, K.-J. Jang, K. Karalis, H. J. Kim, L. MacQueen, R. Mahmoodian, Engineered in vitro disease models, Annu. Rev. Pathol. Mech. Dis. 10 (2015) 195-262.
[26] J. Vanderburgh, J. A. Sterling, S. A. Guelcher, 3D printing of tissue engineered constructs for in vitro modeling of disease progression and drug screening, Ann. Biomed. Eng. 45 (2017) 164-179.
[27] S. Piluso, Y. Li, F. Abinzano, R. Levato, L. Moreira Teixeira, M. Karperien, J. Leijten, R. van Weeren, J. Malda, Mimicking the Articular Joint with In Vitro Models, Trends Biotechnol. 37 (2019) 1063-1077.
[28] J. Erndt-Marino, P. Diaz-Rodriguez, M. S. Hahn, Initial In Vitro Development of a Potassium-Based Intra-Articular Injection for Osteoarthritis, Tissue Eng. Part A. 24 (2018) 1390-1392.
[29] C. Stüdle, Q. Vallmajó-Martín, A. Haumer, J. Guerrero, M. Centola, A. Mehrkens, D. J. Schaefer, M. Ehrbar, A. Barbero, I. Martin, Spatially confined induction of endochondral ossification by functionalized hydrogels for ectopic engineering of osteochondral tissues, Biomaterials. 171 (2018) 219-229.
[30] I. Chiesa, R. D. Gesù, K. J. Overholt, R. Gottardi, A mesoscale 3D culture system for native and engineered biphasic tissues: application to the osteochondral unit, in: Organ—Device, Springer, 2022: pp. 267-281.
[31] B. Prabhakarpandian, M.-C. Shen, J. B. Nichols, I. R. Mills, M. Sidoryk-Wegrzynowicz, M. Aschner, K. Pant, SyM-BBB: A Microfluidic Blood Brain Barrier Model, Lab. Device. 13 (2013) 1093-1101.
[32] M. Egger, G. E. M. Tovar, E. Hoch, A. Southan, Gelatin methacrylamide as coating material in cell culture, Biointerphases. 11 (2016) 021007.
[33] S. Arora, A. Srinivasan, C. M. Leung, Y.-C. Toh, Bio-mimicking Shear Stress Environments for Enhancing Mesenchymal Stem Cell Differentiation, Curr. Stem Cell Res. Ther. 15 (2020) 414-427.
[34] J. B. Lian, M. D. McKee, A. M. Todd, L. C. Gerstenfeld, Induction of bone-related proteins, osteocalcin and osteopontin, and their matrix ultrastructural localization with development of chondrocyte hypertrophy in vitro, J. Cell. Biochem. 52 (1993) 206-219.
[35] L. Yang, K. Y. Tsang, H. C. Tang, D. Chan, K. S. E. Cheah, Hypertrophic chondrocytes can become osteoblasts and osteocytes in endochondral bone formation, Proc. Natl. Acad. Sci. U.S.A 111 (2014) 12097-12102.
[36] Chondrocytes transdifferentiate into osteoblasts in endochondral bone during development, postnatal growth and fracture healing in mice-PubMed, (n.d.).
[37] C. Guo, L. Yuan, J. Wang, F. Wang, X.-K. Yang, F. Zhang, J. Song, X. Ma, Q. Cheng, G. Song, Lipopolysaccharide (LPS) induces the apoptosis and inhibits osteoblast differentiation through JNK pathway in MC3T3-E1 cells, Inflammation. 37 (2014) 621-631.
[38] X. Li, B. Chang, B. Wang, W. Bu, L. Zhao, J. Liu, L. Meng, L. Wang, Y. Xin, D. Wang, Q. Tang, C. Zheng, H. Sun, Rapamycin promotes osteogenesis under inflammatory conditions, Mol. Med. Rep. 16 (2017) 8923-8929.
[39] I. Chiesa, C. De Maria, A. Lapomarda, G. M. Fortunato, F. Montemurro, R. Di Gesù, R. S. Tuan, G. Vozzi, R. Gottardi, Endothelial cells support osteogenesis in an in vitro vascularized bone model developed by 3D bioprinting, Biofabrication. 12 (2020) 025013.
[40] A. Pirosa, R. Gottardi, P. G. Alexander, D. Puppi, F. Chiellini, R. S. Tuan, An in vitro chondro-osteo-vascular triphasic model of the osteochondral complex, Biomaterials. 272 (2021) 120773.
[41] A. Dickhut, E. Gottwald, E. D. Steck, C. Heisel, W. Richter, Chondrogenesis of mesenchymal stem cells in gel-like biomaterials in vitro and in vivo., Front. Biosci. J. Virtual Libr. 13 (2008) 4517-4528.
[42] L. Sun, X. Wang, D. L. Kaplan, A 3D cartilage-inflammatory cell culture system for the modeling of human osteoarthritis, Biomaterials. 32 (2011) 5581-5589.
[43] A. Pirosa, E. B. Tankus, A. Mainardi, P. Occhetta, L. Dönges, C. Baum, M. Rasponi, I. Martin, A. Barbero, Modeling In Vitro Osteoarthritis Phenotypes in a Vascularized Bone Model Based on a Bone-Marrow Derived Mesenchymal Cell Line and Endothelial Cells, Int. J. Mol. Sci. 22 (2021) 9581.
[44] T. Cao, K.-H. Ho, S.-H. Teoh, Scaffold Design and in Vitro Study of Osteochondral Coculture in a Three-Dimensional Porous Polycaprolactone Scaffold Fabricated by Fused Deposition Modeling, Tissue Eng. 9 (2003) 103-112.
[45] T. P. Lozito, P. G. Alexander, H. Lin, R. Gottardi, A. Cheng, R. S. Tuan, Three-dimensional osteochondral microtissue to model pathogenesis of osteoarthritis, Stem Cell Res. Ther. 4 (2013) S6.
[46] Z. Liu, S. Mackay, D. M. Gordon, J. D. Anderson, D. W. Haithcock, C. J. Garson, G. J. Tearney, G. M. Solomon, K. Pant, B. Prabhakarpandian, S. M. Rowe, J. S. Guimbellot, Co-cultured microfluidic model of the airway optimized for microscopy and micro-optical coherence tomography imaging, Biomed. Opt. Express. 10 (2019) 5414-5430.
[47] S. Halldorsson, E. Lucumi, R. Gómez-Sjöberg, R. M. T. Fleming, Advantages and challenges of microfluidic cell culture in polydimethylsiloxane devices, Biosens. Bioelectron. 63 (2015) 218-231.
[48] M. Jang, A. Kleber, T. Ruckelshausen, R. Betzholz, A. Manz, Differentiation of the human liver progenitor cell line (HepaRG) on a microfluidic-based biodevice, J. Tissue Eng. Regen. Med. 13 (2019) 482-494.
[49] P. Occhetta, M. Centola, B. Tonnarelli, A. Redaelli, I. Martin, M. Rasponi, High-Throughput Microfluidic Platform for 3D Cultures of Mesenchymal Stem Cells, Towards Engineering Developmental Processes, Sci. Rep. 5 (2015) 10288.
[50] R. R. Besser, A. C. Bowles, A. Alassaf, D. Carbonero, R. Maciel, M. Saporta, A. Agarwal, A Chemically Defined Common Medium for Culture of C2C12 Skeletal Muscle and Human Induced Pluripotent Stem Cell Derived Spinal Spheroids, Cell. Mol. Bioeng. 13 (2020) 605-619.
[51] K. K. Lee, H. A. McCauley, T. R. Broda, M. J. Kofron, J. M. Wells, C. I. Hong, Human stomach-on-a-device with luminal flow and peristaltic-like motility, Lab. Device. 18 (2018) 3079-3085.
[52] K. K. P. Lee, T. Matsu-Ura, A. E. Rosselot, T. R. Broda, J. M. Wells, C. I. Hong, An integrated microfluidic bubble pocket for long-term perfused three-dimensional intestine-on-a-device model, Biomicrofluidics. 15 (2021) 014110.
[53] W. Lorenz, C. Buhrmann, A. Mobasheri, C. Lueders, M. Shakibaei, Bacterial lipopolysaccharides form procollagen-endotoxin complexes that trigger cartilage inflammation and degeneration: implications for the development of rheumatoid arthritis, Arthritis Res. Ther. 15 (2013) R111.
[54] Z. Y. Huang, T. Stabler, F. X. Pei, V. B. Kraus, Both systemic and local lipopolysaccharide (LPS) burden are associated with knee OA severity and inflammation, Osteoarthritis Cartilage. 24 (2016) 1769-1775.
[55] H. A. Kim, M.-L. Cho, H. Y. Choi, C. S. Yoon, J. Y. Jhun, H. J. Oh, H.-Y. Kim, The catabolic pathway mediated by Toll-like receptors in human osteoarthritic chondrocytes, Arthritis Rheum. 54 (2006) 2152-2163.
[56] A. Perl, Activation of mTOR (mechanistic target of rapamycin) in rheumatic diseases, Nat. Rev. Rheumatol. 12 (2016) 169-182.
[57] B. Caramés, A. Hasegawa, N. Taniguchi, S. Miyaki, F. J. Blanco, M. Lotz, Autophagy activation by rapamycin reduces severity of experimental osteoarthritis, Ann. Rheum. Dis. 71 (2012) 575-581.
[58] Z. Gu, W. Tan, J. Ji, G. Feng, Y. Meng, Z. Da, G. Guo, Y. Xia, X. Zhu, G. Shi, C. Cheng, Rapamycin reverses the senescent phenotype and improves immunoregulation of mesenchymal stem cells from MRL/lpr mice and systemic lupus erythematosus patients through inhibition of the mTOR signaling pathway, Aging. 8 (2016) 1102-1114.
[59] Y. Liu, X. Li, A. Jin, Rapamycin Inhibits Nf-KB Activation by Autophagy to Reduce Catabolismin Human Chondrocytes, J. Investig. Surg. Off. J. Acad. Surg. Res. 33 (2020) 861-873.
[60] F. Li, V. X. Truong, H. Thissen, J. E. Frith, J. S. Forsythe, Microfluidic Encapsulation of Human Mesenchymal Stem Cells for Articular Cartilage Tissue Regeneration, ACS Appl. Mater. Interfaces. 9 (2017) 8589-8601.
[61] P. Occhetta, A. Mainardi, E. Votta, Q. Vallmajo-Martin, M. Ehrbar, I. Martin, A. Barbero, M. Rasponi, Hyperphysiological compression of articular cartilage induces an osteoarthritic phenotype in a cartilage-on-a-device model, Nat. Biomed. Eng. 3 (2019) 545-557.
[62] C. A. Paggi, B. Venzac, M. Karperien, J. C. H. Leijten, S. Le Gac, Monolithic microfluidic platform for exerting gradients of compression on cell-laden hydrogels, and application to a model of the articular cartilage, Sens. Actuators B Chem. 315 (2020) 127917.
[63] X. Shi, J. Zhou, Y. Zhao, L. Li, H. Wu, Gradient-regulated hydrogel for interface tissue engineering: steering simultaneous osteo/chondrogenesis of stem cells on a device, Adv. Healthc. Mater. 2 (2013) 846-853.
[64] X. Bao, Z. Li, H. Liu, K. Feng, F. Yin, H. Li, J. Qin, Stimulation of chondrocytes and chondroinduced mesenchymal stem cells by osteoinduced mesenchymal stem cells under a fluid flow stimulus on an integrated microfluidic device, Mol. Med. Rep. 17 (2018) 2277-2288.
[65] T. M. Griffin, F. Guilak, The Role of Mechanical Loading in the Onset and Progression of Osteoarthritis, Exerc. Sport Sci. Rev. 33 (2005) 195-200.
[66] C. Mondadori, R. Visone, M. Rasponi, A. Redaelli, M. Moretti, S. Lopa, Development of an organotypic microfluidic model to reproduce monocyte extravasation process in the osteoarthritic joint, Osteoarthritis Cartilage. 26 (2018) S122.

1. A microfluidic in vitro osteochondral (OC) device, comprising:
a synovial chamber;
a cartilage chamber adjacent to and porously coupled with the synovial chamber;
a bone chamber adjacent to and porously coupled with the cartilage chamber;
a vasculature circulation chamber adjacent to and porously coupled with the bone chamber;
wherein a first porous wall is positioned between the synovial chamber and the cartilage chamber, a second porous wall is positioned between the cartilage chamber and the bone chamber, and a third porous wall is positioned between the bone chamber and the vasculature circulation chamber,
which is configured as a microfluidic in vitro model of an in vivo osteochondral region.

2. The microfluidic in vitro OC device of claim 1, wherein at least one of:
the synovial chamber is dimensioned as a fluid reservoir or a microvasculature structure that is configured to be coupled to a fluidic network with one or more pumps and optionally one or more media reservoirs;
the vasculature circulation chamber is dimensioned as a physiological microvasculature structure that is configured to be coupled to the fluidic network with one or more pumps and optionally one or more media reservoirs;
the cartilage chamber is dimensioned as a physiological cartilage region, which may optionally be configured to be coupled to the fluidic network with one or more pumps and optionally one or more media reservoirs; or
the bone chamber is dimensioned as a physiological bone region, which may optionally be configured to be coupled to the fluidic network with one or more pumps and optionally one or more media reservoirs.

3. The microfluidic in vitro OC device of claim 1, comprising in order:
the synovial chamber;
the first porous wall;
the cartilage chamber;
the second porous wall;
the bone chamber;
the third porous wall; and
the vasculature circulation system; or
reverse order thereof.

4. The microfluidic in vitro OC device of claim 1, comprising in order:
the synovial chamber having a width in a range from about 50 microns to about 2000 microns;
the first porous wall having a width in a range from about 20 microns to about 100 microns;
the cartilage chamber having a width in a range from about 50 microns to about 2000 microns;
the second porous wall having a width in a range from about 20 microns to about 100 microns;
the bone chamber having a width in a range from about 50 microns to about 2000 microns;
the third porous wall having a width in a range from about 20 microns to about 100 microns;
the vasculature circulation chamber having a width in a range from about 50 microns to about 2000 microns; and
the synovial chamber, cartilage chamber, bone chamber, and vasculature circulation chamber can have a height that ranges from about 10 microns to about 1000 microns.

5. The microfluidic in vitro OC device of claim 4, comprising in order:
the synovial chamber having a length in a range from about 1 mm to about 50 mm;
the first porous wall having a length in a range from about 1 mm to about 50 mm;
the cartilage chamber having a length in a range from about 1 mm to about 50 mm;
the second porous wall having a length of about 1 mm to about 50 mm;
the bone chamber having a length in a range from about 1 mm to about 50 mm;
the third porous wall having a length in a range from about 1 mm to about 50 mm; and
the vasculature circulation chamber having a length in a range from about 1 mm to about 50 mm.

6. The microfluidic in vitro OC device of claim 5, wherein at least one of:
each porous wall includes a plurality of pore channels that have a width that ranges from about 3 microns to about 8 microns and a height that ranges from about 6 microns to about 10 microns; or
each pore channel is spaced from about 5 microns to about 75 microns apart from another pore channel.

7. The microfluidic in vitro OC device of claim 1, wherein:
the synovial chamber is either devoid of cells or includes immune cells, such as PBMCs or macrophages;
the cartilage chamber includes chondrocyte cells, chondroblast cells, and/or chondroclast cells derived from human mesenchymal stem cells or that are primary cells;
the bone chamber includes osteoblast cells, osteoclast cells, and/or osteocyte cells derived from human mesenchymal stem cells or that are primary cells; and
the vasculature circulation chamber includes vascular endothelial cells,
where in cells are in culture with or without a hydrogel matrix.

8. The microfluidic in vitro OC device of claim 7, wherein at least one of the chondrocyte cells, chondroblast cells, chondroclast cells, osteoblast cells, osteoclast cells, or osteocyte cells are differentiated from human mesenchymal cells within the respective chamber of the in vitro OC device.

9. A microfluidic in vitro OC system comprising:
the microfluidic in vitro OC device of claim 1; and
at least one pump configured for pumping fluid through the microfluidic in vitro OC device.

10. A microfluidic in vitro OC system comprising:
the microfluidic in vitro OC device of claim 1;
at least one camera device configured to be positioned to image at least one of the synovial chamber, cartilage chamber, bone chamber, or vasculature circulation chamber; and
a computing system operably coupled with the at least one camera device to receive image data.

11. The microfluidic in vitro OC system of claim 10, wherein the computing system includes a non-transitory memory device having instructions to obtain data from the at least one camera device and determine at least one trans-OC transport barrier property of the microfluidic in vitro OC device or at least one trans-OC transport property of a test agent, wherein the trans-OC transport barrier property is a measurement of inhibition of transport of an agent across the cartilage chamber and/or bone chamber, and the trans-OC transport property of a test agent is a measurement of traversal of the test agent across the cartilage chamber and/or bone chamber.

12. A method of studying an osteochondral environment, comprising:
providing the microfluidic in vitro OC device of claim 1 comprising:
the cartilage chamber includes chondrocyte cells, chondroblast cells, and/or chondroclast cells differentiated from human mesenchymal stem cells;
the bone chamber includes osteoblast cells, osteoclast cells, and/or osteocyte cells differentiated from human mesenchymal stem cells; and
the vasculature circulation chamber includes vascular endothelial cells;
measuring a first condition of the microfluidic in vitro OC device at a first time point;
measuring a second condition of the in vitro OC device at a subsequent time point; and
determining a change in condition of the in vitro OC device from the first condition to the second condition.

13. The method of studying the OC of claim 12, further comprising at least one of:
measuring a barrier function property of the bone chamber and/or cartilage chamber;
imaging the synovial chamber, cartilage chamber, bone chamber, or vasculature circulation chamber through a viewing window of the device;
viewing images in real time of the synovial chamber, cartilage chamber, bone chamber, or vasculature circulation chamber through a display screen of a computing system;
monitoring a cellular morphological change of cells in at least one of the synovial chamber, cartilage chamber, bone chamber, or vasculature circulation chamber;
monitoring a physiochemical change of cells in at least one of the synovial chamber, cartilage chamber, bone chamber, or vasculature circulation chamber;
monitoring a cellular gene expression changes of cells in at least one of the synovial chamber, cartilage chamber, bone chamber, or vasculature circulation chamber;
monitoring a cellular transcriptome change of cells in at least one of the synovial chamber, cartilage chamber, bone chamber, or vasculature circulation chamber;
monitoring a cellular proteome change of cells in at least one of the synovial chamber, cartilage chamber, bone chamber, or vasculature circulation chamber;
monitoring inflammation of cells in at least one of the synovial chamber, cartilage chamber, bone chamber, or vasculature circulation chamber;
monitoring changes in biomolecules in response to test agents of cells in at least one of the synovial chamber, cartilage chamber, bone chamber, or vasculature circulation chamber;
monitoring viability of cells in at least one of the synovial chamber, cartilage chamber, bone chamber, or vasculature circulation chamber; or
measuring transport across at least one of the cartilage chamber and/or bone chamber of at least one of nutrients, xenobiotics, small molecules, lipids, liposomes, polymers, particles, toxins, antibodies, or combinations thereof.

14. The method of claim 12, further comprising introducing a test agent into the device into one of the synovial chamber or vasculature circulation chamber, wherein the first condition is prior to introducing a test agent into the device and the second condition is after introducing the test agent into the device.

15. A method of studying transport of a test agent across a OC, comprising:
providing the microfluidic in vitro OC device of claim 1 comprising:
the cartilage chamber includes chondrocyte cells, chondroblast cells, and/or chondroclast cells;
the bone chamber includes osteoblast cells, osteoclast cells, and/or osteocyte cells; and
the vasculature circulation chamber includes vascular endothelial cells;
providing a test agent to an input chamber selected from the synovial chamber, cartilage chamber, bone chamber, or vasculature circulation chamber; and
monitoring transport of the test agent across at least one of the cartilage chamber or the bone chamber.

16. The method of studying transport of the test agents of claim 15, further comprising at least one of:
determining an amount of test agent crossing the bone chamber and/or cartilage chamber and comparing the amount of test agent that crossed the bone chamber and/or cartilage chamber with the administered amount of the test agent introduced into the microfluidic in vitro OC device;
sampling the vasculature circulation chamber for the test agent and quantifying the transport of the test agent across the bone chamber and/or cartilage chamber into the vasculature circulation chamber; or
sampling the synovial chamber for the test agent and quantifying the transport of the test agent across the bone chamber and/or cartilage into the synovial chamber.

17. The method of studying transport of the test agents of claim 15, further comprising evaluating transport of differently sized particles by:
injecting a plurality of different test agents having a plurality of different sizes into the synovial chamber and/or vasculature circulation chamber;
imaging the in vitro OC device;
analyzing images of the in vitro OC device to identify the plurality of different test agents; and
determining a size of test agent or size range of test agent of the plurality of test agents located in the synovial chamber, cartilage chamber, bone chamber, and/or vasculature circulation chamber.

18. The method of studying transport of the test agents of claim 17, further comprising determining at least one of:
a size of test agent or size range of test agents capable of transporting from the synovial chamber into one of the cartilage chamber, bone chamber, and/or vasculature circulation chamber;
a lipophilicity of test agent or lipophilicity range of test agents capable of transporting from the synovial chamber into one of the cartilage chamber, bone chamber, and/or vasculature circulation chamber;
a physiological charge of test agent or charge range capable of transporting from the synovial chamber into one of the cartilage chamber, bone chamber, and/or vasculature circulation chamber;
a size of test agent or size range of test agents capable of transporting from the vasculature circulation chamber into one of the cartilage chamber, bone chamber, and/or synovial chamber;
a lipophilicity of test agent or lipophilicity range of test agents capable of transporting from the vasculature circulation chamber into one of the cartilage chamber, bone chamber, and/or synovial chamber; or
a physiological charge of test agent or charge range capable of transporting from the vasculature circulation chamber into one of the cartilage chamber, bone chamber, and/or synovial chamber.

19. The method of studying transport of the test agents of claim 18, further comprising evaluating permeability of the in vitro OC device by:
injecting one or more test agents into the synovial chamber and/or vasculature circulation chamber, and optionally the bone chamber and/or cartilage chamber;
imaging the microfluidic in vitro OC device;
analyzing images of the microfluidic in vitro OC device to identify locations of the test agent at defined time points, and optionally determine amounts of each test agent in each chamber; and
determining a permeability of the in vitro OC device for the one or more test agents.

20. The method of studying transport of the test agents of claim 19, further comprising determining a permeability index as a ratio of optical intensity measurements of the synovial chamber with the vasculature circulation chamber, individually for one or more agents.

21. The method of studying transport of the test agents of claim 15, further comprising evaluating whether the test agent modifies permeability or structural integrity or morphology of cells in the bone chamber and/or the cartilage chamber by:
determining an initial value of a first property of cells in the bone chamber and/or the cartilage chamber;
introducing the test agent into the microfluidic in vitro OC device;
determining a subsequent value of the first property of the cells of the bone chamber and/or the cartilage chamber; and
determining a difference between the initial value and the subsequent value of the first property of the cells in the bone chamber and/or cartilage chamber.

22. The method of studying transport of the test agents of claim 21, further comprising determining a health consequence of the test agent modulating the bone chamber by correlating the difference between the initial value and the subsequent value and a phenotypic state, which phenotypic state may or may not be a disease state or disorder state.

23. A method of differentiating cells comprising:
providing the in vitro OC device of claim 1;
introducing first human mesenchymal stem cell into the cartilage chamber;
introducing a chondrogenic differentiation medium into the cartilage chamber with the first human mesenchymal stem cells;
incubating the first human mesenchymal stem cells with the chondrogenic differentiation medium sufficiently to differentiate into at least one of chondrocyte cells, chondroblast cells, and/or chondroclast cells;
incubating second human mesenchymal stem cells with osteogenic differentiation medium sufficiently to differentiate into at least one of osteoblast cells, osteoclast cells, and/or osteocyte cells;
obtaining the differentiated at least one of osteoblast cells, osteoclast cells, and/or osteocyte cells in the bone chamber; and
introducing vascular endothelial cells into the vasculature chamber.

24. The method of claim 23, further comprising:
incubating the second human mesenchymal stem cells with osteogenic differentiation medium sufficiently to differentiate into at least one of osteoblast cells, osteoclast cells, and/or osteocyte cells in a separate cell culture chamber;
obtaining the differentiated at least one of osteoblast cells, osteoclast cells, and/or osteocyte cells from the separate cell culture chamber; and
introducing the differentiated at least one of osteoblast cells, osteoclast cells, and/or osteocyte cells into the bone chamber.

25. The method of claim 23, further comprising:
incubating the vascular endothelial cells with growth medium in a second separate cell culture chamber; and
obtaining the vascular endothelial cells from the second separate cell culture chamber.

26. The method of claim 23, wherein the first human mesenchymal stem cells are introduced into the cartilage chamber.

27. The method of claim 23, further comprising introducing growth medium to the first human mesenchymal stem cells before introducing the chondrogenic differentiation medium.

28. The method of claim 23, wherein at least partially differentiated at least one of osteoblast cells, osteoclast cells, and/or osteocyte cells are introduced into the bone channel.

29. The method of claim 23, wherein the vascular endothelial cells are introduced into the vasculature circulation channel.

30. The method of claim 23, further comprising:
introducing the first human mesenchymal stem cells and growth media into the cartilage chamber at an initial time point;
introducing the chondrogenic differentiation medium into the fluidic circulation channel and/or cartilage chamber from 1 to 20 days after the initial time point;
introducing the differentiated at least one of osteoblast cells, osteoclast cells, and/or osteocyte cells into the bone chamber from 1-20 days after the initial time point;
introducing the vascular endothelial cells into the vasculature circulation chamber from 1-20 days after the initial time point;
supplying growth medium to the synovial chamber and/or cartilage chamber after cessation of chondrogenic differentiation medium;
supplying growth medium to the bone chamber and/or vasculature chamber after introduction of the differentiated at least one of osteoblast cells, osteoclast cells, and/or osteocyte cells into the bone chamber and/or introduction of the vascular endothelial cells into the vasculature circulation chamber.

31. A method of assaying an in vitro osteochondral model, comprising:
performing the method of claim 30; and
performing an assay with the in vitro OC device by changing a condition within at least one chamber, wherein the condition is at least one of presence or absence of a test agent, positive control agent, and/or negative control agent.
